(19) United States
(12) Patent Application Publication
(10) Pub. No.: US 2011/0202284 A1
(43) Pub. Date: Aug. 18, 2011

(54) NOVEL GROUPS OF BIOMARKERS FOR DIAGNOSING ALZHEIMER'S DISEASE

(76) Inventors: Cristopher McReynolds, Woodside, CA (US); David M. Rocke, Davis, CA (US); Lynn Kozma, Sunol, CA (US); Anton Wyss-Coray, Stanford, CA (US)

(21) Appl. No.: 13/025,061

(22) Filed: Feb. 10, 2011

Related U.S. Application Data
(60) Provisional application No. 61/303,199, filed on Feb. 10, 2010.

(51) Int. Cl. G06F 19/00; G01N 33/48

(52) U.S. Cl. 702/19

(57) ABSTRACT

The inventors have discovered sets of proteinaceous biomarkers which can be measured in biological fluid samples to diagnosis or aid in the diagnosis of Alzheimer's disease and distinguish AD samples from non-demented samples.

### CROSS-REFERENCED TO RELATED APPLICATIONS

This application claims priority to U.S. Provisional Patent Application Ser. No. 61/303,199, filed on Feb. 10, 2010, which is hereby incorporated by reference in its entirety.

### BACKGROUND OF THE INVENTION

There are more than 70 conditions that produce clinical dementia in humans, of which Alzheimer's Disease (AD) is the most common. The epidemiological findings for incidences (per 100,000) within the USA in the early 1990s show that the incidences of AD are 0 per 100,000, 3.3 per 100,000, and 99.9 per 100,000 between the ages of 40-49, 50-59, and 60-69, respectively. See Knopman et al., Neurology 62:506-8 (2004).

Alzheimer's Disease (AD) is a neurodegenerative disease of the central nervous system associated with progressive memory loss, cognitive deficits, and dementia resulting in impaired functions in daily living and behavioral symptoms. Two neuropathological hallmarks are observed in AD patients at autopsy: extracellular plaques and intracellular tangles in the hippocampus, cerebral cortex, and other areas of the brain essential for cognitive function. Plaques are formed mostly from the deposition of amyloid beta (“Aβ”), a peptide derived from amyloid precursor protein (“APP”). Filamentous tangles are formed from paired helical filaments composed of neurofilament and hyperphosphorylated tau protein, a microtubule-associated protein. It is not clear, however, whether these two pathological changes are merely associated with the disease or truly involved in the degenerative process. To date, genetic studies have identified three genes that cause autosomal dominant, early-onset AD: amyloid precursor protein (“APP”), presenilin 1 (“PS1”), and presenilin 2 (“PS2”). A fourth gene, apolipoprotein E (“ApoE”), is the strongest and most common genetic risk factor for AD, but does not necessarily cause it. All mutations associated with APP and PS proteins can lead to an increase in the production of Aβ peptides, specifically the more amyloidogenic form, Aβ₄₂. In addition to genetic influences on amyloid plaque and intracellular tangle formation, environmental factors (e.g., cytokines, neurotoxins, etc.) may also play important roles in the development and progression of AD.

It is estimated that the prevalence of AD in the United States (U.S.) is predicted to increase from 4.5 million people to 14 million people by 2050 in the absence of the discovery of preventative treatments. This accounts for roughly 85% of dementias in the U.S. See Mandavilli, Nat. Med. 12:747-51(2006).

Given the magnitude of the public health problem posed by AD, considerable research efforts have been undertaken to elucidate the etiology of AD, as well as to identify biomarkers (secreted proteins or metabolites) that can be used to diagnose and/or predict whether a person is likely to develop AD. Such biomarkers can be of utility to the development of novel therapeutics at a number of stages in the drug development process. For example, identification of these biomarkers at research and preclinical stages provide the values of more rigorous pathway analysis, target identification, target validation, mechanism association, and biochemical modeling; identification of these biomarkers at Phases I-III stages enables potentially smaller, faster, and more cleanly focused clinical trials by screening out those patients with other conditions that mimic the clinical presentation of AD such as psychiatric disorders, reactions to prescription drugs, or prolonged alcohol abuse, thus improving the probability of success of clinical trials and greater efficiency in patient selection and surrogate endpoints; and identification of these biomarkers at the regulatory approval and reimbursement and launch/life cycle management ultimately could be useful for faster regulatory approval, higher reimbursement, faster time to clinical guidelines, greater uptake rate, greater market penetration, and lower marketing costs.

With regards to biomarkers for AD, the proteins amyloid beta and tau are probably the most well-characterized. Research has shown that cerebrospinal fluid (“CSF”) samples from AD patients contain higher than normal amounts of tau, which is released as neurons degenerate, and lower than normal amounts of beta amyloid, presumably because it is trapped in the brain in the form of amyloid plaques. Because these biomarkers are released into CSF, a lumbar puncture (or “spinal tap”) is required to obtain a sample for testing.

We have previously demonstrated that peripheral signaling proteins may also be useful as biomarkers for AD. See Ray et al., Nat. Med. 13:1359-1362 (2007). This was based on the hypothesis that changes in the extracellular milieu within the brain in response to the disease state of the patient would be reflected in the periphery and, therefore, measurable in the blood. A growing body of literature supports the notion that there are perturbations in the immune and/or inflammatory mechanisms associated with AD within the brain itself, and also measurable in the periphery.

While there are currently more than 20 drugs on the market for the treatment of AD (see, e.g., Delagarza, Clin. Pharmcol. 68:1365-72 (2003)), most are thought to treat only symptoms, rather than being truly disease-modifying. Consequently, there is room for improvement in the development and commercialization of novel therapeutics to meet this growing medical need. Currently there are more than 100 compounds at various stages of development in several pharmaceutical companies: 111 compounds are at preclinical stage, 22 compounds are at Phase I clinical trial, 30 compounds are at Phase II clinical trial, 14 compounds are at Phase III clinical trial, 1 compound is at pre-registration stage, 2 compounds are at registered stage, and 22 compounds have already been launched (Halteras Consulting).

For most of the compounds currently in development, the molecular target is known, representing more than 30 unique protein targets. However, clinical trials for novel therapeutics for AD continue to be hampered by the lack of clear, objective, and highly accurate tests for the disease. Further, current criteria for diagnosis of AD rely largely on the exclusion of other causes for cognitive dysfunction and include a battery of neurophysiological, blood, and neuroimaging tests that are time-consuming and expensive. A definite diagnosis is currently only possible upon autopsy.

A number of U.S. patents and applications have been published relating to methods for diagnosing AD, including U.S. Pat. Nos. 4,728,605, 5,874,312, 6,027,896, 6,114,133, 6,130,048, 6,210,895, 6,358,681, 6,451,547, 6,461,831, 6,465,195, 6,475,161, and 6,495,335, and U.S. patent application Ser. Nos. 12/093,431, 11/580,405, 11/148,595, 12/480,222, and 12/577,082, and U.S. Pat. No. 7,598,049. Additionally, a number of reports in the scientific literature relate to certain biochemical markers and their correlation/association with AD, including Fahnestock et al., J. Neural. Transm. Suppl. (62):241-52 (2002); Masliah et al., 1195, Neurobiol. Aging 16(4):549-56; Power et al., Dement. Geriatr. Cogn. Disord. 12(2):167-70 (2001); and Burbach et al., J. Neurosci. 24(10):2421-30 (2004).

All patents, patent applications, publications, and references cited herein are incorporated by reference in their entirety.

### BRIEF SUMMARY OF THE INVENTION

The inventors have discovered sets or groups of biochemical markers, present in biological fluid samples from individuals, including from the blood or the plasma of individuals, which are differentially altered in individuals with Alzheimer's disease (“AD”) versus non-demented control (“NDC”) individuals. Accordingly, these sets of biomarkers (“AD diagnosis biomarkers”) may be used to diagnose or aid in the diagnosis of AD.

Various embodiments are described with reference to certain aspects of the invention. However, it is to be understood that the various embodiments described herein may be used in other aspects of the invention as described herein, as will be apparent to one of ordinary skill in the art.

The present invention provides a method of diagnosing or aiding diagnosis of Alzheimer's disease (“AD”), comprising: a) measuring levels of a set of AD diagnosis biomarkers in a biological fluid sample in an individual, wherein the set comprises at least three AD diagnosis biomarkers, wherein the at least three AD diagnosis biomarkers are selected from the group consisting of: ACE (angiotensin converting enzyme or CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand); b) implementing a scoring algorithm; c) generating a score based on the measured levels of the AD diagnosis biomarkers; and d) comparing the score with a cutoff score, wherein the diagnosis of AD is made or aided by determining whether the score is above, equal to, or below the cutoff score, and wherein the method has an accuracy of at least about 65% in classifying the individual as either having Alzheimer's disease or nondemented. The present invention also provides a computer implemented method of diagnosing or aiding diagnosis of Alzheimer's disease (“AD”) comprising: a) implementing a scoring algorithm; b) generating a score based on measured levels of a set of AD diagnosis biomarkers in a biological fluid sample in an individual; and c) comparing the score with a cutoff score, wherein the diagnosis of AD is made or aided by determining whether the score is above, equal to, or below the cutoff score, wherein the set of AD diagnosis biomarkers comprises at least three AD diagnosis biomarkers, wherein the at least three AD diagnosis biomarkers are selected from the group consisting of: ACE (angiotensin converting enzyme or CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand), and wherein the method has an accuracy of at least about 65% in classifying the individual as either having Alzheimer's disease or nondemented. In some embodiments, the method comprises diagnosing whether the individual has AD. In some embodiments, the method comprises aiding in the diagnosis of whether the individual has AD. In some embodiments, the individual is classified as having Alzheimer's disease if the score is equal to or above the cut-off score, or classified as nondemented if the score is below the cut-off score. In some embodiments, the individual is classified as having Alzheimer's disease if the score is above the cut-off score or classified as nondemented if the score is equal to or below the cut-off score. In some embodiments, the individual is classified as having Alzheimer's disease if the score is equal to or below the cut-off score, or classified as nondemented if the score is above the cut-off score. In some embodiments, the individual is classified as having Alzheimer's disease if the score is below the cut-off score, or classified as nondemented if the score is equal to or above the cut-off score. In some embodiments, the set of AD diagnosis biomarkers comprises at least four AD diagnosis biomarkers, wherein the at least four AD diagnosis biomarkers are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand). In some embodiments, the set of AD diagnosis biomarkers comprises at least five AD diagnosis biomarkers, wherein the at least five AD diagnosis biomarkers are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand). In some embodiments, the set of AD diagnosis biomarkers comprises at least six AD diagnosis biomarkers, wherein the at least six AD diagnosis biomarkers are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand). In some embodiments, the set of AD diagnosis biomarkers comprises at least seven AD diagnosis biomarkers, wherein the at least seven AD diagnosis biomarkers are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand). In some embodiments, the set of AD diagnosis biomarkers comprises at least eight AD diagnosis biomarkers, wherein the at least eight AD diagnosis biomarkers are selected from the group consisting of: ACE (angiotensin converting enzyme, CD 143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand). In some embodiments, the set of AD diagnosis biomarkers comprises at least nine AD diagnosis biomarkers, wherein the at least nine AD diagnosis biomarkers are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand). In various embodiments, the set of AD diagnosis biomarkers comprises at least ten AD diagnosis biomarkers, wherein the at least ten AD diagnosis biomarkers are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand). In some embodiments, the set of AD diagnosis biomarkers comprises at least eleven AD diagnosis biomarkers, wherein the at least eleven AD diagnosis biomarkers are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand). In some embodiments, the set of AD diagosis biomakers comprises at least one of the following AD diagnosis biomarkers: ACE, cortisol, Apo E, MCP-3, and MMP-9. In some embodiments, the set of AD diagosis biomakers comprises at least two of the following AD diagnosis biomarkers: ACE, cortisol, Apo E, MCP-3, and MMP-9 (e.g. ACE and cortisol, ACE and Apo E, ACE and MCP-3, ACE and MMP-9, cortisol and Apo E, cortisol and MCP-3, cortisol and MMP-9, Apo E and MCP-3, Apo E and MMP-9, or MCP-3 and MMP-9). In some embodiments, the set of AD diagosis biomakers comprises at least three of the following AD diagnosis biomarkers: ACE, cortisol, Apo E, MCP-3, and MMP-9 (e.g. ACE, cortisol, and Apo E; ACE, cortisol, and MCP-3; ACE, cortisol, and MMP-9; ACE, Apo E, and MCP-3; ACE, Apo E, and MMP-9; ACE, MCP-3, and MMP-9; cortisol, Apo E, and MCP-3; cortisol, Apo E, and MMP-9; cortisol, MCP-3, and MMP-9; or Apo E, MCP-3, and MMP-9). In some embodiments, the set of AD diagosis biomakers comprises at least four of the following AD diagnosis biomarkers: ACE, cortisol, Apo E, MCP-3, and MMP-9 (e.g. ACE, cortisol, Apo E, and MCP-3; ACE, cortisol, Apo E, and MMP-9; ACE, cortisol, MCP-3, and MMP-9; ACE, Apo E, MCP-3, and MMP-9; or cortisol, Apo E, MCP-3, and MMP-9). In some embodiments, the set of AD diagosis biomakers comprises at least the following AD diagnosis biomarkers: ACE, cortisol, Apo E, MCP-3, and MMP-9. In some embodiments, the set of AD diagosis biomakers comprises ACE. In some embodiments, the set of AD diagosis biomakers comprises cortisol. In some embodiments, the set of AD diagosis biomakers comprises Apo E. In some embodiments, the set of AD diagosis biomakers comprises MCP-3. In some embodiments, the set of AD diagosis biomakers comprises MMP-9. The set of AD diagnosis biomarkers may comprise any set shown in Table 3. In some embodiments, the set of AD diagnosis biomarkers further comprise one or more additional biomarkers. In various embodiments, the method has an accuracy of at least about 66%, at least about 67%, at least about 68%, at least about 69%, at least about 70%, at least about 71%, at least about 72%, at least about 74%, at least about 75%, at least about 76%, at least about 77%, at least about 78%, at least about 79%, at least about 80% in classifying the individual as either having Alzheimer's disease or nondemented. In some embodiments, the cutoff score is generated by a scoring algorithm. In some embodiments, the cutoff score is determined by the sample distribution of the sample set used to develop the scoring algorithm. In some embodiments, the cutoff score is based on measured levels of the AD diagnosis biomarkers in a control population and a population having AD. In some embodiments, the control population is a non-demented control (NDC) population. In some embodiments, the control population is a healthy non-demented control (NDC) population. In some embodiments, the control population is a non-demented control (NDC) population comprising healthy and non-healthy individuals. In some embodiments, the control population and/or the population having AD is selected from an age-matched population and/or a sex-matched population. It is to be understood that “in a biological fluid sample” includes measuring each AD diagnosis biomarker in a single biological fluid sample, or measuring the AD diagnosis biomarkers in two or more different samples (e.g., each biomarker measured in a separate sample, some biomarkers measured in one sample and the rest measured in another sample, etc.). In some embodiments, the biological fluid sample is a peripheral biological fluid sample. In some embodiments, the peripheral biological fluid sample is blood, serum, or plasma. In some embodiments, the peripheral biological fluid sample is plasma. In some embodiments, the plasma is EDTA plasma. In some embodiments, the individual is a human. In some embodiments, the scoring algorithm comprises a method selected from the group consisting of: Significance Analysis of Microarrays, Tree Harvesting, CART, MARS, Self Organizing Maps, Frequent Item Set, Bayesian networks, Prediction Analysis of Microarray (PAM), SMO, Simple Logistic Regression, Logistic Regression, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, Ordinal Classifier, Sparse Linear Programming (SPLP), Sparse Logistic Regression (SPLR), Elastic NET, Support Vector Machine, Prediction of Residual Error Sum of Squares (PRESS), and combinations thereof. In some embodiments, the scoring algorithm is implemented by a computer. In some embodiments, the score is determined by the formula: (e^{sum})/(1+e^{sum}), wherein the sum is determined by the formula: Intercept+ΣCoefficient(AD diagnosis biomarkerᵢ)*ln [AD diagnosis biomarkerᵢ+0.005], wherein i is 1 to N, N is the number of AD diagnosis biomarkers used, and [AD diagnosis biomarker] is the actual measured level of an AD diagnosis biomarker. In some embodiments, the measuring is performed by a sandwich antibody array assay. In some embodiments, the score can be combined with one or more cognitive assessment tools and/or clinical observations in making the diagnosis or in aiding the diagnosis of AD. In some embodiments, the measured levels and/or values are obtained by measuring the levels and/or values of the AD diagnosis biomarkers in the sample(s), while in other embodiments the measured values and/or levels are obtained from a third party.

In another aspect of the invention is a kit comprising at least one reagent specific for each of at least three AD diagnosis biomarkers in a biological fluid sample in an individual, wherein the at least three AD diagnosis biomarkers are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand), wherein the at least three AD biomarkers are useful in a method for diagnosis or aiding diagnosis of AD, and wherein the method has an accuracy of at least about 65% in classifying the individual as either having Alzheimer's disease or nondemented. In various embodiments, provided is a kit comprising at least one reagent specific for each of at least four, at least five, at least six, at least seven, at least eight, at least nine, at least ten, or at least eleven AD diagnosis biomarkers in a biological fluid sample in an individual, wherein the at least four, at least five, at least six, at least seven, at least eight, at least nine, at least ten, or at least eleven AD diagnosis biomarkers are selected from the group consisting of: ACE (angiotensin converting enzyme, CD 143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand), wherein the at least four, at least five, at least six, at least seven, at least eight, at least nine, at least ten, or at least eleven AD biomarkers are useful in a method for diagnosis or aiding diagnosis of AD, and wherein the method has an accuracy of at least about 65% in classifying the individual as either having Alzheimer's disease or nondemented. In some embodiments, the kit further comprises instructions for carrying out a method as described herein. In some embodiments, the at least one reagent specific for each AD diagnosis biomarker is an antibody, or fragment thereof, that is specific for the AD diagnosis biomarker. In some embodiments, the at least one reagent is useful for a sandwich antibody array assay. In some embodiments, the kit includes one or more reagents which can detect common variants of one or more of the AD diagnosis biomarkers, wherein a common variant indicates a protein that is expressed in at least 5 percent or more of the population in industrialized nations. In some embodiments, the kit further comprises at least one reagent specific for one or more additional biomarkers. In some embodiments, the kit comprises at least one reagent specific for a biomarker that measures sample characteristics. In some embodiments, the reagent specific for a biomarker that measures sample characteristics is an AD diagnosis biomarker. In some embodiments, the reagent specific for a biomarker that measures sample characteristics is not an AD diagnosis biomarker.

In another aspect of the invention is a surface comprising attached thereto, at least one reagent specific for each of at least three AD diagnosis biomarkers in a biological fluid sample in an individual, wherein the at least three AD diagnosis biomarkers are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand), wherein the at least three AD biomarkers are useful in a method for diagnosis or aiding diagnosis of AD, and wherein the method has an accuracy of at least about 65% in classifying the individual as either having Alzheimer's disease or nondemented. In various embodiments, provided is a surface comprising attached thereto at least one reagent specific for each of at least four, at least five, at least six, at least seven, at least eight, at least nine, at least ten, or at least eleven AD diagnosis biomarkers in a biological fluid sample in an individual, wherein the at least four, at least five, at least six, at least seven, at least eight, at least nine, at least ten, or at least eleven AD diagnosis biomarkers are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand), wherein the at least four, at least five, at least six, at least seven, at least eight, at least nine, at least ten, or at least eleven AD biomarkers are useful in a method for diagnosis or aiding diagnosis of AD, and wherein the method has an accuracy of at least about 65% in classifying the individual as either having Alzheimer's disease or nondemented. In some embodiments, provided herein are surfaces wherein said reagents specific for the AD diagnosis biomarkers are antibodies, or fragments thereof, that are specific for the AD diagnosis biomarkers. In some embodiments, the surface is useful in a sandwich antibody array assay. In some embodiments, the surface comprises at least one reagent specific for a biomarker that measures sample characteristics. In some embodiments, the reagent specific for a biomarker that measures sample characteristics is an AD diagnosis biomarker. In some embodiments, the reagent specific for a biomarker that measures sample characteristics is not an AD diagnosis biomarker. The surfaces may be used in any of the methods described herein.

In another aspect of the invention is a combination comprising a surface as described herein and a peripheral biological fluid sample from an individual. In some embodiments, said individual is at least 40, 45, 55, 60, 65, 70, 75, 80, or 85 years of age.

In another aspect of the invention is a computer readable format comprising measured levels and/or values and/or reference levels and/or values as obtained by a method described herein.

In another aspect of the invention is a machine-readable storage medium including a data storage material encoded with machine-readable data wherein a machine programmed with instructions for using said data may perforin a method or portion of a method as described herein. The data may include, for example, measured levels and/or values and/or reference levels and/or values as obtained by a method described herein.

In another aspect of the invention is an apparatus (e.g. computer system) having logic operable to perform operations corresponding to a method described herein. For example, a computer system may be programmed with instructions for using an algorithm as described herein for classifying an individual as either AD or NDC.

In another aspect of the invention is a computer-readable storage medium comprising instructions for performing a method described herein. In some embodiments, provided is a computer readable storage medium comprising instructions for diagnosing or aiding diagnosis of Alzheimer's disease (“AD”) comprising: a) implementing a scoring algorithm; b) generating a score based on measured levels of a set of AD diagnosis biomarkers in a biological fluid sample in an individual; and c) comparing the score with a cutoff score, wherein the diagnosis of AD is made or aided by determining whether the score is above, equal to, or below the cutoff score, wherein the set of AD diagnosis biomarkers comprises at least three AD diagnosis biomarkers, wherein the at least three AD diagnosis biomarkers are selected from the group consisting of: ACE (angiotensin converting enzyme or CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand), and wherein the method has an accuracy of at least about 65% in classifying the individual as either having Alzheimer's disease or nondemented. In various embodiments, the set of AD diagnosis biomarkers comprises at least four, at least five, at least six, at least seven, at least eight, at least nine, at least ten, or at least eleven AD diagnosis biomarkers, wherein the at least four, at least five, at least six, at least seven, at least eight, at least nine, at least ten, or at least eleven AD diagnosis biomarkers are selected from the group consisting of: ACE (angiotensin converting enzyme, CD 143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand).

In one aspect is a method of diagnosis or aiding diagnosis of Alzheimer's disease (“AD”), comprising comparing measured levels of a set of AD diagnosis biomarkers in a biological fluid sample from an individual seeking a diagnosis for AD to one or more reference levels, wherein the set comprises at least three AD diagnosis biomarkers selected from the group consisting of: ACE (angiotensin converting enzyme or CD 143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand); wherein the method has an accuracy of at least about 65% in classifying the individual as either having Alzheimer's disease or nondemented. In various embodiments, the set of AD diagnosis biomarkers comprises at lease four, at least five, at least six, at least seven, at least eight, at least nine, at least ten, at least eleven AD diagnosis biomarkers. In some embodiments, the method comprises comparing the measured levels to a standard concentration curve for each AD diagnosis biomarker and calculating the estimated concentration by fitting to the curve. In some embodiments, said biological fluid sample is a peripheral biological fluid sample. In some embodiments, said peripheral biological fluid sample is blood, serum or plasma. In some embodiments, said peripheral biological fluid sample is serum or plasma. In some embodiments, the method further comprises obtaining the measured levels of said AD biomarkers in said biological fluid sample. In some embodiments, the individual is a human. In some embodiments, the measured levels are normalized. In some embodiments, the reference level(s) are normalized In some embodiments, the reference level is a cutoff score for use in a scoring algorithm. In some embodiments, the reference level(s) are obtained from measured values of the at least three biomarkers from samples in the blood of human individuals with AD. In some embodiments, the reference level(s) are obtained from measured values of the at least three biomarkers from samples in the blood of human individuals without AD. In some embodiments, the group of individuals without AD is a control population selected from: a population of healthy non-demented individuals, and a population of non-demented individuals comprising healthy and non-healthy individuals. In some embodiments, the control population is age- and/or sex-matched. In some embodiments, comparing comprises a method selected from the group consisting of: Significance Analysis of Microarrays, Tree Harvesting, CART, MARS, Self Organizing Maps, Frequent Item Set, Bayesian networks, Prediction Analysis of Microarray (PAM), SMO, Simple Logistic Regression, Logistic Regression, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, Support Vector Machine, and Ordinal Classifier. In some embodiments, comparing comprises a method comprising Logisitic Regression. In some embodiments, the method further comprises: formulating a decision tree; and using the decision tree for classification of the sample from the individual seeking AD diagnosis, wherein the classification aids the diagnosis of AD. In some embodiments, using the decision tree for classification of the sample is implemented by a computer. In some embodiments, the diagnosis of AD is aided by determining a difference between the measured levels of the at least three AD diagnosis biomarkers to the reference levels of the at least three biomarkers wherein the difference meets or exceeds a statistically significant difference between normalized measured values of the at least three AD diagnosis biomarkers in the blood samples from individuals without AD and individuals with AD, wherein the statistically significant difference indicates a diagnosis of AD. In some embodiments, the measured levels are normalized. In some embodiments, the references level(s) are normalized. In some embodiments, the measured values are normalized. In some embodiments, the method further comprises: formulating a decision tree comprising statistically significant differences in normalized measured levels of the AD diagnosis biomarkers wherein the statistically significant differences are determined from normalized measured values of the plurality of AD diagnosis biomarkers in the blood samples in the group of individuals with AD and the group of individuals without AD; and using the decision tree for classification of the blood sample from the individual seeking AD diagnosis, wherein the classification aids the diagnosis of AD. In some embodiments, the method of aiding diagnosis of Alzheimer's disease (“AD”), comprises comparing normalized measured levels of at the least three AD diagnosis biomarkers in a blood sample from a human individual seeking a diagnosis for AD to reference levels for the at least three biomarkers in the blood sample, wherein the reference levels are obtained from normalized measured values of the at least three biomarkers from samples in the blood of human individuals without AD, whereby the diagnosis of AD is aided by determining a difference between the normalized measured levels of the at least three AD diagnosis biomarkers to the reference levels of the at least three biomarkers from non-AD samples wherein the difference meets or exceeds a statistically significant difference between normalized measured values of the at least three AD diagnosis biomarkers in the blood samples from individuals without AD and individuals with AD, wherein the statistically significant difference indicates a diagnosis of AD. In some embodiments, the reference levels for the at least three biomarkers are obtained by a method comprising: determining measured levels of the at least three biomarkers in normal individuals with a Mini Mental State Examination (MMSE) score greater than 27, having a statistically significant difference from normalized measured levels of the at least three biomarkers in AD subjects with MMSE score of 27 and below. In some embodiments, the reference levels for the at least three biomarkers are obtained by a method comprising: determining measured levels of the at least three biomarkers in non-demented individuals, having a statistically significant difference from measured levels of the at least three biomarkers in AD individuals, wherein the individuals are classified as non-demented or AD by clinical diagnosis. In some embodiments, concentrations are determined based on the measured levels. In some embodiments, aiding the diagnosis of AD further comprises one or more clinical diagnostic methods comprising taking patient histories, administering memory tests, attributing a MMSE score, administering psychological tests, or ruling out temporary or permanent conditions that may explain memory loss. In some embodiments, determining the statistically significant difference associated with a diagnosis of AD comprises: determining a mean, median, or shrunken centroid value of normalized measured values of the at least three AD diagnosis biomarkers in the blood samples from a group of individuals with AD; determining a mean, median, or shrunken centroid value of normalized measured values of the at least three AD diagnosis biomarkers in the blood samples from a group of individuals without AD; and finding a statistically significant difference between the mean, median, or shrunken centroid values of the normalized measured values of the at least three AD diagnosis biomarkers in the blood samples between the two groups. In some embodiments, the method further comprises: formulating a decision tree comprising statistically significant differences in measured values of AD diagnosis biomarkers wherein the statistically significant differences are determined from measured values of the plurality of AD diagnosis biomarkers in the blood samples in the group of individuals with AD and the group of individuals without AD; and using the decision tree for classification of the blood sample from the individual seeking AD diagnosis, wherein the classification aids the diagnosis of AD. In some embodiments, the measured values of the plurality of AD diagnosis biomarkers in the blood samples from the group of individuals with AD and the group of individuals without AD form statistically significant differences in measured values for learning samples. In some embodiments, the method further comprises: comparing the statistically significant differences in normalized measured levels of AD diagnosis biomarkers in the blood sample from the individual seeking AD diagnosis, with the statistically significant differences in normalized measured values for learning samples.

It is understood that aspects and embodiments of the invention described herein include “comprising”, “consisting of” and/or “consisting essentially of” aspects and embodiments.

### BRIEF DESCRIPTION OF THE DRAWINGS

FIG. 1 shows marker frequency for all models above 65% accuracy.

FIG. 2 shows marker frequency for all unique models above 65% accuracy (multiple cutoffs eliminated).

### DETAILED DESCRIPTION OF THE INVENTION

The inventors have discovered sets of biochemical markers (collectively termed “AD diagnosis biomarkers”) useful for diagnosis of AD and aiding in the diagnosis of AD. The AD diagnosis biomarkers are present in biological fluids of individuals. In some examples, the AD diagnosis biomarkers are present in peripheral biological fluids (e.g., plasma) of individuals, allowing collection of samples by procedures that are relatively non-invasive, particularly as compared to the lumbar puncture procedure commonly used to collect cerebrospinal fluid samples.

The inventors assert that measuring concentrations of particular sets of secreted biomarkers (e.g., 3-11 biomarkers) in the blood is a more sensitive method for the determination of disease than measuring an absolute concentration of any single biochemical marker. A composite or array embodying the use of the sets of biomarkers as described herein, consisting of, for example, antibodies bound to a solid support or protein bound to a solid support, for the detection of inflammation and injury response biomarkers associated with AD.

### DEFINITIONS

As used herein, the terms “Alzheimer's patient,” “AD patient,” “individual diagnosed with AD,” and “population having AD” all refer to an individual who has been diagnosed with AD or has been given a probable diagnosis of Alzheimer's disease (AD).

As used herein, the phrase “AD diagnosis biomarker” refers to a biomarker that is useful in differentiating AD patients from non-demented patients.

The term “AD diagnosis biomarker polynucleotide”, as used herein, refers to any of: a polynucleotide sequence encoding an AD diagnosis biomarker, the associated trans-acting control elements (e.g., promoter, enhancer, and other gene regulatory sequences), and/or mRNA encoding the AD diagnosis biomarker.

As used herein, methods for “aiding diagnosis” refer to methods that assist in making a clinical determination regarding the presence, or nature, of the AD, and may or may not be conclusive with respect to the definitive diagnosis. Accordingly, for example, a method of aiding diagnosis of AD can comprise measuring the levels of the set of AD diagnosis biomarkers in a biological sample from an individual. Optionally, one or more additional methods may be used in conjunction with the methods of aiding diagnosis of AD as described herein, for example, various cognitive assessment tools and/or clinical observations.

As used herein, “biological fluid sample” encompasses a variety of fluid sample types obtained from an individual and can be used in a diagnostic assay. The definition encompasses, e.g., blood, cerebral spinal fluid (CSF), urine and other liquid samples of biological origin. The definition also includes samples that have been manipulated in any way after their procurement, such as by treatment with reagents (e.g., EDTA plasma), solubilization, or enrichment for certain components, such as proteins or polynucleotides.

As used herein, the term “peripheral biological fluid sample” refers to a biological fluid sample that is not derived from the central nervous system (i.e., is not a CSF sample) and includes blood samples and other biological fluids not derived from the CNS.

A “blood sample” is a biological sample which is derived from blood, preferably peripheral (or circulating) blood. A blood sample may be, for example, whole blood, plasma or serum.

An “individual” is a mammal, more preferably a human. Mammals include, but are not limited to, humans, primates, farm animals, sport animals, rodents, and pets.

“Sensitivity” and “specificity” are statistical measures of the performance of a binary classification test. Sensitivity measures the proportion of actual positives which are correctly identified as such (e.g. the percentage of individuals having Alzheimer's who are identified as having the condition). Specificity measures the proportion of negatives which are correctly identified (e.g. the percentage of non-demented individuals who are identified as not having Alzheimer's). A true positive indicates an Alzheimer's sample correctly identified as Alzheimer's. A false positive indicates a non-demented sample incorrectly identified as Alzheimer's. A true negative indicates a non-demented sample correctly identified as non-demented. A false negative indicates an Alzheimer's sample incorrectly identified as non-demented. As used herein, “Accuracy”=(number of True Positives+number of True Negatives)/(all Positives+all Negatives). As used herein, “Specificity”=number of True Negatives/(number of True Negatives+number of False Positives). As used herein, “Sensitivity”=number of True Positives/(number of True Positives+number of False Negatives). In cases where the number of Alzheimer's samples is equal to the number of non-demented samples, the Accuracy is the average of the Sensitivity and Specificity.

As used herein, “a”, “an”, and “the” can mean singular or plural (i.e., can mean one or more) unless indicated otherwise.

As used herein, reference to “about” a value or parameter includes (and describes) embodiments that are directed to that value or parameter per se. For example, description referring to “about X” includes description of “X.”

### Biomarker Information

The names and abbreviations of AD diagnosis biomarkers described herein, and references are listed in Table 1. UniProt ID numbers are provided in parentheses.

TABLE 1
AD Diagnosis Biomarkers

| Biomarker Name (UniProt ID) | Abbreviation(s) | References |
|---|---|---|
| Angiotensin converting enzyme (P12821) | ACE, CD 143 | Zou et al., Helbecque et al., |
| | | Wright and Harding |
| Alpha 1 antitrypsin (Q86U18) | | Song et al. |
| Apolipoprotein AI (P02647) | Apo AI, Apo A1 | Song et al., Paula-Lima et |
| | | al. |
| Apolipoprotein CIII (P02656) | Apo CIII | Wada |
| Apolipoprotein E (P02649) | Apo E | Wada, Zhang et al. |
| C reactive protein (P02741) | CRP | Mancinella et al., Roberts |
| | | et al. |
| Cortisol (none) | | Huang et al. |
| Fibroblast growth factor-4 (P08620) | FGF-4 | |
| Monocyte chemotactic protein-3 or Monocyte | MCP-3 | |
| chemotactic protein-3 (P80098) | | |
| Matrix matelloproteinase-9 (P14780) | MMP-9 | Bruno et al. |
| Tumor necrosis factor-related apoptosis- | TRAIL R3 | |
| inducing ligand receptor 3 (O14798) | | |

### REFERENCES

J Zhang, I Sokal, E R Peskind, J F Quinn, J Jankovic, C Kenney, K A Chung, S P Millard, J G Nutt, T J Montine, “CSF multianalyte profile distinguishes Alzheimer and Parkinson diseases,” Am J Clin Pathol 129:526-9, 2008.
H Wada, “Analyses of serum concentrations of apolipoproteins in the demented elderly,” Intern Med 39:220-2, 2000.
K Zou, T Maeda, A Watanabe, J Liu, S Liu, R Oba, Y Satoh, H Komano, M Michikawa, “Abeta42-to-Abeta40- and angiotensin-converting activities in different domains of angiotensin-converting enzyme,” J Biol Chem 284:31914-20, 2009.
J W Wright and J W Harding, “The brain RAS and Alzheimer's disease,” Exp Neurol epub September 2009.
N Helbecque, V Codron, D Cottel, P Amouyel, “An age effect on the association of common variants of ACE with Alzheimer's disease,” Neurosci Lett 461:181-4, 2009.
F Song, A Poljak, G A Smythe, P Sachdev, “Plasma biomarkers for mild cognitive impairment and Alzheimer's disease,” Brain Res Rev, 61:69-80, 2009.
A C Paula-Lima, M A Tricerri, J Brito-Moreira, T R Bomfim, F F Oliveira, M H Magdesian, L T Grinberg, R Panizzutti, S T Ferreira, “Human apolipoprotein A-I binds amyloid-beta and prevents Abeta-induced neurotoxicity,” Int J Biochem Cell Biol, 41:1361-70, 2009.
A Mancinella, M Mancinella, G Carpinteri, A Bellomo, C Fossati, V Gianturco, A Iori, E Ettorre, G Troisi, V Marigliano, “Is there a relationship between high C-reactive protein (CRP) levels and dementia?” Arch Gerontol Geriatr, 49 Suppl 1:185-94, 2009.
R O Roberts, Y E Geda, D S Knopman, B F Boeve, T J Christianson, V S Pankratz, I J Kullo, E G Tangalos, R J Ivnik, R C Petersen, “Association of C-reative protein with mild cognitive impairment,” Alzheimers Dement, 5:398-405, 2009.
C W Huang, CC Lui, W N Chang, C H Lu, Y L Wang, CC Chang, “Elevated basal cortisol level predicts lower hippocampal volume and cognitive decline in Alzheimer's disease,” J Clin Neurosci, 16:1283-6, 2009.
M A Bruno, E J Mufson, J Wuu, A C Cuello, “Increased matrix metalloproteinase 9 activity in mild cognitive impairment,” J Neuropathol Exp Neurol, epub November 2009.

### METHODS OF THE INVENTION

Provided herein are methods of diagnosing AD or aiding diagnosis of AD.

For example, a method of aiding diagnosis of Alzheimer's disease (“AD”) may comprise: a) measuring a level of each of at least three AD diagnosis biomarkers in a biological fluid sample in an individual, wherein the at least three AD diagnosis biomarkers are selected from the group consisting of: ACE (angiotensin converting enzyme or CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand); b) implementing a scoring algorithm; c) generating a score based on the measured levels of the AD diagnosis biomarkers; and d) comparing the score with a cutoff score, wherein the diagnosis of AD is aided by determining whether the score is above, equal to, or below the cutoff score; wherein the method has an accuracy of at least about 65% in classifying the individual as either having Alzheimer's disease or nondemented.

The accuracy, sensitivity, and/or specificity of the AD diagnosis methods of the instant invention utilizing sets of biomarkers as described herein are generally enhanced over the use of a single biomarker. The AD diagnosis biomarkers for use in the methods described herein include various sets (e.g., a set of at least three, at least four, at least five, at least six, at least seven, at least eight, at least nine, at least ten, or at least eleven AD diagnosis biomarkers that are selected from the group consisting of: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand). These sets described herein provide an accuracy of at least about 65% in methods for classifying an individual as either having AD or nondemented, when combined with particular cutoff scores (see e.g. Table 3 below which describes particular sets of AD diagnosis biomarkers and cutoffs which have a 65% total accuracy). In some embodiments, these sets of biomarkers do not include any additional biomarkers. In some embodiments, these sets of biomarkers may further include one or more additional biomarkers (e.g. one or more AD diagnosis biomarkers). Additional biomarkers are described in U.S. patent application Ser. Nos. 12/093,431, 11/580,405, 11/148,595, 12/480,222, and 12/577,082, and U.S. Pat. No. 7,598,049.

### Measuring Levels of AD Diagnosis Biomarkers

A primary measurement of a level of a particular AD diagnosis biomarker may be a measurement of the quantity of AD diagnosis biomarker itself (quantitative data), such as by detecting the number of AD diagnosis biomarker molecules in the sample. The measurement of a level of a particular AD diagnosis biomarker may be a secondary measurement (a measurement from which the quantity of the biomarker can be but not necessarily deduced, such as a measure of enzymatic activity (when the biomarker is an enzyme) or a measure of mRNA coding for the biomarker).

As used herein, the AD diagnosis biomarkers are selected biomarkers including but not limited to secreted proteins or metabolites present in a person's biological fluids (that is, a biological fluid sample, for example, a peripheral biological fluid sample), such as for example, blood, including whole blood, plasma (e.g., EDTA plasma), serum, or other biological fluid. A blood sample may include, for example, various cell types present in the blood including platelets, lymphocytes, polymorphonuclear cells, macrophages, and erythrocytes.

Although some assay formats allow testing of biological fluid samples without prior processing of the sample, most biological fluid samples are processed prior to testing. Processing generally takes the form of elimination of cells (nucleated and non-nucleated), such as erythrocytes, leukocytes, and platelets in blood samples, and may also include the elimination of certain proteins, such as certain clotting cascade proteins from blood or other standard means of protein separation, enrichment, or purification. In some examples, the biological fluid sample is collected in a container comprising EDTA.

In some embodiments, AD diagnosis biomarker levels are measured using an affinity-based measurement technology. “Affinity” as relates to an antibody is a term well understood in the art and means the extent, or strength, of binding of antibody to the binding partner, such as an AD diagnosis biomarker as described herein (or epitope thereof). Affinity may be measured and/or expressed in a number of ways known in the art, including, but not limited to, equilibrium dissociation constant (KD or Kd), apparent equilibrium dissociation constant (KD' or Kd'), and IC50 (amount needed to effect 50% inhibition in a competition assay; used interchangeably herein with “I50”). It is understood that, for purposes of this invention, an affinity is an average affinity for a given population of antibodies which bind to an epitope. Values of KD' reported herein in terms of mg IgG per ml or mg/ml indicates mg Ig per ml of serum, although plasma can be used.

Affinity-based measurement technology utilizes a molecule that specifically binds to the AD diagnosis biomarker being measured (an “affinity reagent,” such as an antibody or aptamer), although other technologies, such as spectroscopy-based technologies (e.g., matrix-assisted laser desorption ionization-time of flight, or MALDI-TOF, spectroscopy) or assays measuring bioactivity (e.g., assays measuring mitogenicity of growth factors) may be used.

Affinity-based technologies include antibody-based assays (immunoassays) and assays utilizing aptamers (nucleic acid molecules which specifically bind to other molecules), such as ELONA. Additionally, assays utilizing both antibodies and aptamers are also contemplated (e.g., an sandwich format assay utilizing an antibody for capture and an aptamer for detection).

If immunoassay technology is employed, any immunoassay technology which can quantitatively or qualitatively measure the level of an AD diagnosis biomarker in a biological sample may be used. Suitable immunoassay technology includes, for example, radioimmunoassay, immunofluorescent assay, enzyme immunoassay, chemiluminescent assay, ELISA (e.g., sandwich ELISA), immuno-PCR, immuno-infrared, western blot assay, and Luminex Platform utilizing color-coded beads and antibodies.

Likewise, aptamer-based assays which can quantitatively or qualitatively measure the level of an AD diagnosis biomarker in a biological sample may be used in the methods of the invention. Generally, aptamers may be substituted for antibodies in nearly all formats of immunoassay, although aptamers allow additional assay formats (such as amplification of bound aptamers using nucleic acid amplification technology such as PCR (U.S. Pat. No. 4,683,202) or isothermal amplification with composite primers (U.S. Pat. Nos. 6,251,639 and 6,692,918).

A wide variety of affinity-based assays are known in the art Affinity-based assays will utilize at least one epitope derived from the AD diagnosis biomarker of interest, and many affinity-based assay formats utilize more than one epitope (e.g., two or more epitopes are involved in “sandwich” format assays; at least one epitope is used to capture the biomarker, and at least one different epitope is used to detect the biomarker).

Affinity-based assays may be in competition or direct reaction formats, utilize sandwich-type formats, and may further be heterogeneous (e.g., utilize solid supports) or homogenous (e.g., take place in a single phase) and/or utilize or immunoprecipitation. Most assays involve the use of labeled affinity reagent (e.g., antibody, polypeptide, or aptamer); the labels may be, for example, enzymatic, fluorescent, chemiluminescent, radioactive, or dye molecules. In another approach, all the proteins in the biological sample can be labeled using standard protein chemistry techniques and the labeled biomarkers are captured by the affinity reagents arrayed on a solid support. Assays which amplify the signals from the probe are also known; examples of which are assays which utilize biotin and avidin, and enzyme-labeled and mediated immunoassays, such as ELISA and ELONA assays. Herein, in the examples referred to as “quantitative data”, the biomarker concentrations were obtained using ELISA. Either of the biomarker or reagent specific for the biomarker can be attached to a surface and levels can be measured directly or indirectly.

In a heterogeneous format, the assay utilizes two phases (typically aqueous liquid and solid). Typically an AD diagnosis biomarker-specific affinity reagent is bound to a solid support to facilitate separation of the AD diagnosis biomarker from the bulk of the biological sample. After reaction for a time sufficient to allow for formation of affinity reagent/AD diagnosis biomarker complexes, the solid support or surface containing the antibody is typically washed prior to detection of bound polypeptides. The affinity reagent in the assay for measurement of AD diagnosis biomarkers may be provided on a support (e.g., solid or semi-solid); alternatively, the polypeptides in the sample can be immobilized on a support or surface. Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, microspheres, glass, Protein A beads, magnetic beads, and electrodes. Both standard and competitive formats (with or without chemical modification to enable attachment) for these assays are known in the art. Accordingly, provided herein are complexes comprising a set of AD diagnosis biomarkers as described herein bound to reagents specific for the biomarkers, wherein said reagents are attached to a surface. Also provided herein are complexes comprising a set of AD diagnosis biomarkers as described herein bound to reagents specific for the biomarkers, wherein said biomarkers are attached to a surface.

Array-type heterogeneous assays are suitable for measuring levels of AD diagnosis biomarkers as the methods of the invention utilize multiple AD diagnosis biomarkers. Array-type assays used in the practice of the methods of the invention will commonly utilize a solid substrate with two or more capture reagents specific for different AD diagnosis biomarkers bound to the substrate a predetermined pattern (e.g., a grid). The peripheral biological fluid sample is applied to the substrate and AD diagnosis biomarkers in the sample are bound by the capture reagents. After removal of the sample (and appropriate washing), the bound AD diagnosis biomarkers are detected using a mixture of appropriate detection reagents that specifically bind the various AD diagnosis biomarkers. Binding of the detection reagent is commonly accomplished using a visual system, such as a fluorescent dye-based system. Because the capture reagents are arranged on the substrate in a predetermined pattern, array-type assays provide the advantage of detection of multiple AD diagnosis biomarkers without the need for a multiplexed detection system.

In a homogeneous format the assay takes place in single phase (e.g., aqueous liquid phase). Typically, the biological sample is incubated with an affinity reagent specific for the AD diagnosis biomarker in solution. For example, it may be under conditions that will precipitate any affinity reagent/antibody complexes which are formed. Both standard and competitive formats for these assays are known in the art.

In a standard (direct reaction) format, the level of AD diagnosis biomarker/affinity reagent complex is directly monitored. This may be accomplished by, for example, determining the amount of a labeled detection reagent that forms is bound to AD diagnosis biomarker/affinity reagent complexes. In a competitive format, the amount of AD diagnosis biomarker in the sample is deduced by monitoring the competitive effect on the binding of a known amount of labeled AD diagnosis biomarker (or other competing ligand) in the complex. Amounts of binding or complex formation can be determined either qualitatively or quantitatively.

In some embodiments, sandwich antibody arrays are used in the methods of the invention. In some embodiments, a high sensitivity multiplex sandwich ELISA is used in the methods of the invention, for example, the SearchLight platform utilizing a chemiluminescent readout. In some embodiments, the SearchLight platform is used in the methods of the invention. In some embodiments, the high sensitivity multiplex sandwich ELISA platform (e.g. SearchLight platform) is used to analyze the biomarkers. In some embodiments, a glass array platform that utilizes indirect fluorescence detection is used to analyze one or more biomarkers. In some of these embodiments, the one or more biomarkers analyzed with the glass array platform that utilizes indirect fluorescence detection are control biomarkers. In some embodiments, the Luminex platform is used in the methods of the invention.

The Luminex platform uses up to 100 distinct color-coded bead (e.g., microspheres) sets by varying the ratio of a pair of dyes impregnated into the beads. Each bead set represents a single dye ratio and can be coated with a reagent specific to a particular bioassay. A specific analyte from a sample can then be captured and detected. Such modified bead sets can be combined to enable simultaneous assessment of multiple analytes from the same small sample volume. For the assessment of proteins in complex biological samples, the Luminex platform assay can be configured similarly to a sandwich ELISA assay in which a capture antibody is covalently coupled to the surface of the beads. Following sample incubation and washes, a fluorescently labeled detection antibody is then bound. The complexes are then analyzed in the Luminex instrument which passes the beads in a very narrow stream past a pair of lasers that detect the reporter dye on the detection antibody and the dye ratio present in the bead simultaneously.

Complexes formed comprising AD diagnosis biomarker and an affinity reagent are detected by any of a number of known techniques known in the art, depending on the format of the assay and the preference of the user. For example, unlabelled affinity reagents may be detected with DNA amplification technology (e.g., for aptamers and DNA-labeled antibodies) or labeled “secondary” antibodies which bind the affinity reagent. Alternately, the affinity reagent may be labeled, and the amount of complex may be determined directly (as for dye-(fluorescent or visible), bead-, or enzyme-labeled affinity reagent) or indirectly (as for affinity reagents “tagged” with biotin, expression tags, and the like).

As will be understood by those of skill in the art, the mode of detection of the signal will depend on the exact detection system utilized in the assay. For example, if a radiolabeled detection reagent is utilized, the signal will be measured using a technology capable of quantitating the signal from the biological sample or of comparing the signal from the biological sample with the signal from a reference sample, such as scintillation counting, autoradiography (typically combined with scanning densitometry), and the like. If a chemiluminescent detection system is used, then the signal will typically be detected using a luminometer. Methods for detecting signal from detection systems are well known in the art and need not be further described here.

Optionally, one or more methods may be used for normalization of biomarker levels and/or values, and/or reference levels and/or values. For example, various detection methods and sampling methods may introduce systematic variation that is independent of the underlying biological variation of interest. This is often related directly to errors introduced at sampling, storage, or measurement/analytical assessment and can be dependent on the specific method(s) used. Common normalization techniques for this type of systematic error include, but are not limited to, global methods such as rank normalization, quantile normalization, mean or median normalization, plate/slide/batch normalization as well as nonlinear methods such as LOWESS (locally weighted scatterplot smoothing), B-splines, and Guassian kernel fitting. As will be apparent to those skilled in the art, other similar techniques may also be used.

The biological sample may be divided into a number of aliquots, with separate aliquots used to measure different AD diagnosis biomarkers (although division of the biological sample into multiple aliquots to allow multiple determinations of the levels of the AD diagnosis biomarkers in a particular sample are also contemplated). Alternately the biological sample (or an aliquot therefrom) may be tested to determine the levels of multiple AD diagnosis biomarkers in a single reaction using an assay capable of measuring the individual levels of different AD diagnosis biomarkers in a single assay, such as an array-type assay or assay utilizing multiplexed detection technology (e.g., an assay utilizing detection reagents labeled with different fluorescent dye markers).

It is common in the art to perform ‘replicate’ measurements when measuring AD diagnosis biomarkers. Replicate measurements are ordinarily obtained by splitting a sample into multiple aliquots, and separately measuring the biomarker(s) in separate reactions of the same assay system. Replicate measurements are not necessary to the methods of the invention, but many embodiments of the invention will utilize replicate testing, particularly duplicate and triplicate testing.

### Scoring Algorithm

As described herein, the scoring algorithm is a method that includes, but is not limited to: Significance Analysis of Microarrays, Tree Harvesting (see Hastie et al., Genome Biology 2001, 2:research0003.1-0003.12), CART, MARS, Self Organizing Maps (Kohonen, 1982b, Biological Cybernetics 43(1):59-69), Frequent Item Set (see Agrawal et al., 1993 “Mining association rules between sets of items in large databases.” In Proc. of the ACM SIGMOD Conference on Management of Data, pages 207-216, Washington, D.C., May 1993), Bayesian networks (see Gottardo, Statistical analysis of microarray data, A Bayesian approach. Biostatistics (2001), 1,1, pp 1-37), Prediction Analysis of Microarray (PAM) (see worldwide web at—stat.stanford.edu/˜tibs/PAM/index.html), SMO, Simple Logistic Regression, Logistic Regression, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, Ordinal Classifier, Sparse Linear Programming (SPLP), Sparse Logistic Regression (SPLR), Elastic NET, Support Vector Machine, Prediction of Residual Error Sum of Squares (PRESS), and combinations thereof.

Many of the analysis tools assume that data input for analysis is normally distributed (that is, roughly resembles a bell-shaped curve). For the tools to perform most accurately, the data to be input may be “transformed” to resemble the normal distribution curve. Common methods for normalizing the data for this purpose include (but are not limited to): log transformation (either natural log or the log of any base, most commonly base 10), rank normalization, quantile normalization, mean or median normalization, and/or data scaling, and other similar techniques as would be apparent to one skilled in the art. For some measurement techniques or for some specific biomarkers, the estimated concentrations or direct measurements may be normally distributed and not require any normalization technique for most accurate results with the analysis tool.

In some embodiments, the SAM technique can be used for developing the scoring algorithm The SAM technique assigns a score to each biomarker on the basis of change in expression relative to the standard deviation of repeated measurements (see Tusher et al., 2001, Proc. Natl. Acad. Sci. U.S.A. 98(9):5116-21). For biomarkers with scores greater than an adjustable threshold, the algorithm uses permutations of the repeated measurements to estimate the probability that a particular biomarker has been identified by chance (calculated as a “q-value”), or a false positive rate which is used to measure accuracy. The SAM technique can be carried out using publicly available software called Significance Analysis of Microarrays (see worldwide web at—stat class.stanford.edu/˜tibs/clickwrap/sam.html).

In some embodiments, Prediction Analysis of Microarray (PAM) (see worldwide web: —stat.stanford.edu/˜tibs/PAM/index.html and worldwide web: —stat.stanford.edu/˜tibs/PAM/Rdist/howwork.html) can be used for developing the scoring algorithm.

One embodiment for use of PAM is briefly described. Briefly, the method computes a standardized centroid for each class. This is the average gene expression for each gene in each class divided by the within-class standard deviation for that gene. Nearest centroid classification takes the gene expression profile of a new sample, and compares it to each of these class centroids. The class whose centroid that it is closest to, in squared distance, is the predicted class for that new sample. Nearest shrunken centroid classification makes an important modification to standard nearest centroid classification. It “shrinks” each of the class centroids toward the overall centroid for all classes by an amount called the threshold. This shrinkage consists of moving the centroid towards zero by threshold, setting it equal to zero if it hits zero. For example if threshold was 2.0, a centroid of 3.2 would be shrunk to 1.2, a centroid of −3.4 would be shrunk to −1.4, and a centroid of 1.2 would be shrunk to zero. After shrinking the centroids, the new sample is classified by the usual nearest centroid rule, but using the shrunken class centroids. This shrinkage has two advantages: 1) it can make the classifier more accurate by reducing the effect of noisy genes, 2) it does automatic gene selection. In particular, if a gene is shrunk to zero for all classes, then it is eliminated from the prediction rule. Alternatively, it may be set to zero for all classes except one, and we learn that high or low expression for that gene characterizes that class.

The user decides on the value to use for threshold. Typically one examines a number of different choices. To guide in this choice, PAM does K-fold cross-validation for a range of threshold values. The samples are divided up at random into K roughly equally sized parts. For each part in turn, the classifier is built on the other K−1 parts then tested on the remaining part. This is done for a range of threshold values, and the cross-validated misclassification error rate is reported for each threshold value. Typically, the user would choose the threshold value giving the minimum cross-validated misclassification error rate. What one gets from this is a (typically) accurate classifier that is simple to understand.

In some embodiments, the following method may be used: A semi-supervised prediction analysis is performed using the statistical package PAM 2.3.1 with the statistical tool R. PAM executes a sample classification training routine from expression data via the nearest shrunken centroid procedure to find markers that discriminate best between two classes (e.g. AD vs non-demented control (NDC)). It is to be understood that other classes (e.g. Parkinson's Disease (PD)) may be used in the training set. Then an internal cross-validation is applied by 10-times randomly selecting 90% of the training samples in a class-balanced way to predict each time the class labels on the remaining 10% of samples (10-fold cross-validation). This assesses and minimizes classification errors and avoids over fitting. The obtained minimal number of predictors/markers is then used for a heterogeneity analysis to perform two-class prediction in a test set between two diseased groups.

As used herein, the cutoff score is determined by the scoring algorithm and calculated based on the measured levels of the AD diagnosis biomarkers in a population having (i.e., clinically diagnosed with) AD and a population having (i.e., clinically diagnosed with) no dementia (NDC). In some embodiments, the NDC population and/or the population having AD is selected from an age-matched population and/or a sex-matched population.

Age-matched populations are ideally the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or 10 year increments (e.g. a “5 year increment” group which serves as the source for reference values for a 62 year old individual might include 58-62 year old individuals, 59-63 year old individuals, 60-64 year old individuals, 61-65 year old individuals, or 62-66 year old individuals).

Sex-matched populations ideally have exactly the same percentage of females in each of the two test groups, but approximately sex-matched populations are also acceptable. Approximately sex-matched populations may be within 1-10% difference between the two test groups.

Using an iterative process of successive logistic regression analyses based on the statistical methods as described herein, the parameters of the scoring algorithm, including coefficients for each AD diagnosis biomarker for a set of AD diagnosis biomarkers (e.g., a set of 3-11 biomarkers), and intercept value for a set of AD diagnosis biomarkers, are determined. The coefficients for each AD diagnosis biomarker are indications of the relative weight of each of the biomarkers in making the prediction of AD. The positive and negative values of the coefficients indicate whether the AD diagnosis biomarkers have complementary or off-setting effects. The intercept value is a scaling factor used to adjust the readout of the values of the measured levels of AD diagnosis biomarkers to a range of 0 to 1. Both the coefficients and the intercept value are determined by reviewing the biomarkers and predictions using logistic regression statistical tools. The cutoff score can be determined by reviewing the distribution of calls and comparing them with the true diagnosis, and is selected to provide maximum accuracy in calling either AD or NDC. A balance between sensitivity and specificity may be made. Depending on the desired use of the method, the cutoff score may optimize sensitivity or specificity (e.g. provide a bias towards a relatively lower false positive rate or a bias towards a relatively lower false negative rate for diagnosis of AD). As used herein, the score (or inverse logit) based on the measured levels of a set of AD diagnosis biomarkers in a biological fluid sample is determined by the formula:

Score=(e^{(sum)})/(1+e^{(sum)}),

and the sum is determined by the formula:

Sum=Intercept+ΣCoefficient(AD diagnosis biomarkerᵢ)*ln [AD diagnosis biomarkerᵢ+0.005],

wherein i is 1 to N, and N is the number of AD diagnosis biomarkers used, and [AD diagnosis biomarker] is the actual concentration value of the measured level of an AD diagnosis biomarker.

Accordingly, an individual is diagnosed with AD if the score is less than, less than or equal to, greater than, or greater than or equal to a cutoff score (e.g., 0.5), depending on the particular biomarkers and scoring algorithm (and wherein the individual is classified as nondemented if the score is greater than or equal to, greater than, less than or equal to, or less than, respectively, the cutoff score). As will be known to those skilled in the art, the typical practice is to place the classes on the scoring scale in alphabetical order. Thus, for AD vs. NDC, AD would be below the cutoff and NDC would be above the cutoff but, e.g., for Parkinson's vs. Control, Control would be below the cutoff and Parkinson's would be above it. However, it is to be understood that a scoring algorithm could also be generated which would classify those above the cutoff as AD, and those below the cutoff as NDC. The parameters of the algorithm, including coefficients and the intercept value, can be optimized over time when additional samples are tested. The cutoff score can also be varied to achieve optimized sensitivity or specificity in the assay.

In some embodiments, the cutoff score can be in the range of about 0.25 to about 0.85. In some embodiments, the cutoff score is about 0.1, about 0.2, about 0.3, about 0.4, about 0.5, about 0.6, about 0.7, about 0.8, or about 0.9. In some embodiments, the cutoff score is 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, or 0.85.

In one example, the sum for a set of eleven AD diagnosis biomarkers in a biological fluid sample in an individual can be calculated as:

Sum=Intercept+Coefficient(ACE)*ln [ACE+0.005]+Coefficient(Alpha 1 Antitrypsin)*ln [Alpha 1 Antitrypsin+0.005]+Coefficient(Apo A1)*ln [Apo A1+0.005]+Coefficient(Apo CIII)*ln [Apo CII+0.005]+Coefficient(Apo E)*ln [Apo E+0.005]+Coefficient(CRP)*ln [CRP+0.005]+Coefficient(Cortisol)*ln [Cortisol+0.005]+Coefficient(FGF-4)*ln [FGF-4+0.005]+Coefficient(MCP-3)*ln [MCP-3+0.005]+Coefficient(MMP-9)*ln [MMP-9+0.005]+Coefficient(TRAIL R3)*ln [TRAIL R3+0.005],

and the score for a set of eleven AD diagnosis biomarkers can be calculated as:

Score=(e^{(Intercept+Coefficient(ACE)*ln [ACE+0.005]+Coefficient(Alpha 1 Antitrypsin)*ln [Alpha 1 Antitrypsin+0.005]+Coefficient(Apo A1)*ln [Apo A1+0.005]+Coefficient(Apo CIII)*ln [Apo CII+0.005]+Coefficient(Apo E)*ln [Apo E+0.005]+Coefficient(CRP)*ln [CRP+0.005]+Coefficient(Cortisol)*ln [Cortisol+0.005]+Coefficient(FGF-4)*ln [FGF-4+0.005]+Coefficient(MCP-3)*ln [MCP-3+0.005]+Coefficient(MMP-9)*ln [MMP-9+0.005]+Coefficient(TRAIL R3)*ln [TRAIL R3+0.005])})/(1+e^{(Intercept+Coefficient(ACE)*ln [ACE+0.005]+Coefficient(Alpha 1 Antitrypsin)*ln [Alpha 1 Antitrypsin+0.005]+Coefficient(Apo A1)*ln [Apo A1+0.005]+Coefficient(Apo CIII)*ln [Apo CII+0.005]+Coefficient(Apo E)*ln [Apo E+0.005]+Coefficient(CRP)*ln [CRP+0.005]+Coefficient(Cortisol)*1n [Cortisol+0.005]+Coefficient(FGF-4)*ln [FGF-4+0.005]+Coefficient(MCP-3)*ln [MCP-3+0.005]+Coefficient(MMP-9)*ln [MMP-9+0.005]+Coefficient(TRAIL R3)*ln [TRAIL R3+0.005])}).

The methods described herein may be implemented using any device capable of implementing the methods. Examples of devices that may be used include but are not limited to electronic computational devices, including computers of all types. For example, a typical computing system may be employed to implement processing functionality in embodiments of the invention. Computing systems of this type may be used in clients and servers, for example. Those skilled in the relevant art will also recognize how to implement the invention using other computer systems or architectures. Computing system may represent, for example, a desktop, laptop or notebook computer, hand-held computing device (PDA, cell phone, palmtop, etc), mainframe, server, client, or any other type of special or general purpose computing device as may be desirable or appropriate for a given application or environment. Computing system can include one or more processors. Processor can be implemented using a general or special purpose processing engine such as, for example, a microprocessor, microcontroller or other control logic. For example, a processor is connected to a bus or other communication medium.

Computing system can also include a main memory, such as random access memory (RAM) or other dynamic memory, for storing information and instructions to be executed by processor. Main memory also may be used for storing temporary variables or other intermediate information during execution of instructions to be executed by processor. Computing system may likewise include a read only memory (“ROM”) or other static storage device coupled to bus for storing static information and instructions for a processor.

The computing system may also include information storage system, which may include, for example, a media drive and a removable storage interface. The media drive may include a drive or other mechanism to support fixed or removable storage media, such as a hard disk drive, a floppy disk drive, a magnetic tape drive, an optical disk drive, a CD or DVD drive (R or RW), or other removable or fixed media drive. Storage media, may include, for example, a hard disk, floppy disk, magnetic tape, optical disk, CD or DVD, or other fixed or removable medium that is read by and written to by a media drive. As these examples illustrate, the storage media may include a computer-readable storage medium having stored therein particular computer software or data.

In alternative embodiments, information storage system may include other similar components for allowing computer programs or other instructions or data to be loaded into a computing system. Such components may include, for example, a removable storage unit and an interface, such as a program cartridge and cartridge interface, a removable memory (for example, a flash memory or other removable memory module) and memory slot, and other removable storage units and interfaces that allow software and data to be transferred from the removable storage unit to computing system.

A computing system can also include a communications interface. Communications interface can be used to allow software and data to be transferred between a computing system and external devices. Examples of communications interface can include a modem, a network interface (such as an Ethernet or other NIC card), a communications port (such as for example, a USB port), a PCMCIA slot and card, etc. Software and data transferred via communications interface are in the form of signals which can be electronic, electromagnetic, optical or other signals capable of being received by communications interface. These signals are provided to communications interface via a channel. This channel may carry signals and may be implemented using a wireless medium, wire or cable, fiber optics, or other communications medium. Some examples of a channel include a phone line, a cellular phone link, an RF link, a network interface, a local or wide area network, and other communications channels.

In this document, the terms “computer program product,” “computer-readable medium” and the like may be used generally to refer to physical, tangible media such as, for example, memory, storage devices, or storage unit. These and other forms of computer-readable media may be involved in storing one or more instructions for use by processor, to cause the processor to perform specified operations. Such instructions, generally referred to as “computer program code” (which may be grouped in the form of computer programs or other groupings), when executed, enable the computing system to perform features or functions of embodiments of the present invention. Note that the code may directly cause the processor to perform specified operations, be compiled to do so, and/or be combined with other software, hardware, and/or firmware elements (e.g., libraries for performing standard functions) to do so.

In an embodiment where the elements are implemented using software, the software may be stored in a computer-readable storage medium and loaded into computing system using, for example, a removable storage drive, drive or communications interface. The control logic (in this example, software instructions or computer program code), when executed by the processor, causes the processor to perform the functions of the invention as described herein.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

In various embodiments, the sensitivity (e.g., defining AD as positive and NDC as negative) achieved by the use of the set of AD diagnosis biomarkers in a method for diagnosing or aiding diagnosis of AD is at least about 50%, at least about 55%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In various embodiments, the specificity achieved by the use of the set of AD diagnosis biomarkers in a method for diagnosing or aiding diagnosis of AD is at least about 50%, at least about 55%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In various embodiments, the overall accuracy achieved by the use of the set of AD diagnosis biomarkers in a method for diagnosing or aiding diagnosis of AD is at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%. In some embodiments, the sensitivity and/or specificity are measured against a clinical diagnosis of AD or NDC.

In any of the above methods described herein, the score can be combined with cognitive assessment tools and clinical observations in making the diagnosis or in aiding the diagnosis of AD.

Cognitive assessment tools and clinical observations of relevance include, but are not limited to, a general physical exam including blood work to eliminate vitamin deficiency, medication interactions, or infectious diseases, Apo E genotyping, CSF biomarkers such as but not limited to total tau, phosphor-tau181, A beta 42, and A beta 40, neuroimaging tests such as EEG, PET, MRI, CT scan, PIB, cognition tests such as MMSE, ADAS-Cog (Alzheimer's disease assessment scale cognitive), RAVLT (Ray's auditory verbal learning and memory test), Wechsler adult intelligence scale (and revised), 7MS (Seven minute screen), Mini-Cog, DemTect, Trail making test, etc.

### Kits

The invention provides kits for carrying out any of the methods described herein.

Kits of the invention may comprise at least one reagent specific for each AD diagnosis biomarker in the set, and may further include instructions for carrying out a method described herein. Kits may comprise any set of biomarkers (and/or reagents specific for the set of biomarkers) as described herein. A set of AD diagnosis biomarkers for use in kits provided herein comprises at least three, at least four, at least five, at least six, at least seven, at least eight, at least nine, at least ten, or at least eleven AD diagnosis biomarkers selected from the group consisting of ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand), wherein the at least three, at least four, at least five, at least six, at least seven, at least eight, at least nine, at least ten, or at least eleven AD biomarkers are useful in a method for diagnosis or aiding diagnosis of AD, and wherein the method has an accuracy of at least about 65% in classifying the individual as either having Alzheimer's disease or nondemented. In some embodiments, the set further comprises one or more additional biomarkers. In some embodiments, the kit comprises at least one reagent specific for each AD diagnosis biomarker in the set, wherein the AD diagnosis biomarkers comprise at least three, at least four, at least five, at least six, at least seven, at least eight, at least nine, at least ten, or at least eleven of the following: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand), wherein the at least three, at least four, at least five, at least six, at least seven, at least eight, at least nine, at least ten, or at least eleven AD biomarkers are useful in a method for diagnosis or aiding diagnosis of AD, and wherein the method has an accuracy of at least about 65% in classifying the individual as either having Alzheimer's disease or nondemented. In some embodiments, the kit further comprises at least one reagent specific for one or more additional biomarkers.

Kits comprising a reagent specific for an AD diagnosis biomarker will generally have the reagent enclosed in a container (e.g., a vial, ampoule, or other suitable storage container), although kits including the reagent bound to a substrate (e.g., an inner surface of an assay reaction vessel) are also contemplated. Each reagent may be in a separate container or bound to a separate substrate, or one of more of the reagents may be enclosed within a single container or bound to a single substrate.

In some embodiments, the AD diagnosis biomarker-specific reagent(s) will be labeled with a detectable marker (such as a fluorescent dye or a detectable enzyme), or be modified to facilitate detection (e.g., biotinylated to allow for detection with an avidin- or streptavidin-based detection system). In other embodiments, the AD diagnosis biomarker-specific reagent will not be directly labeled or modified.

Certain kits of the invention will also include one or more agents for detection of bound AD diagnosis biomarker-specific reagent. As will be apparent to those of skill in the art, the identity of the detection agents will depend on the type of AD diagnosis biomarker-specific reagent(s) included in the kit, and the intended detection system. Detection agents include antibodies specific for the AD diagnosis biomarker-specific reagent (e.g., secondary antibodies), primers for amplification of an AD diagnosis biomarker-specific reagent that is nucleotide based (e.g., aptamer) or of a nucleotide ‘tag’ attached to the AD diagnosis biomarker-specific reagent, avidin- or streptavidin-conjugates for detection of biotin-modified AD diagnosis biomarker-specific reagent(s), and the like. Detection systems are well known in the art, and need not be further described here.

A modified substrate or other system for capture of AD diagnosis biomarkers may also be included in the kits of the invention, particularly when the kit is designed for use in a sandwich-format assay. The capture system may be any capture system useful in an AD diagnosis biomarker assay system, such as a multi-well plate coated with an AD diagnosis biomarker-specific reagent, beads coated with an AD diagnosis biomarker-specific reagent, and the like. Capture systems are well known in the art and need not be further described here.

In certain embodiments, kits for use in the methods disclosed herein include the reagents in the form of an array. The array includes different reagents (each reagent specific for a different AD diagnosis biomarker) bound to a substrate in a predetermined pattern (e.g., a grid). Accordingly, the present invention provides arrays comprising reagents for AD diagnosis biomarkers including, but not limited to AD diagnosis biomarkers listed in Table 1. In some embodiments, arrays comprising reagents for a set of AD diagnosis biomarkers comprising at least three, at least four, at least five, at least six, at least seven, at least eight, at least nine, at least ten, or at least eleven of the following: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand). In some embodiments, the array further comprises reagents for one or more additional biomarkers. The localization of the different AD diagnosis biomarker-specific reagents (the “capture reagents”) allows measurement of levels of a number of different AD diagnosis biomarkers in the same reaction. Kits including the reagents in array form are commonly in a sandwich format, so such kits may also comprise detection reagents. Normally, the kit will include different detection reagents, each detection reagent specific to a different AD diagnosis biomarker. The detection reagents in such embodiments are normally reagents specific for the same AD diagnosis biomarkers as the reagents bound to the substrate (although the detection reagents typically bind to a different portion or site on the AD diagnosis biomarker target than the substrate-bound reagents), and are generally affinity-type detection reagents. As with detection reagents for any other format assay, the detection reagents may be modified with a detectable moiety, modified to allow binding of a separate detectable moiety, or be unmodified. Array-type kits including detection reagents that are either unmodified or modified to allow binding of a separate detectable moiety may also contain additional detectable moieties (e.g., detectable moieties which bind to the detection reagent, such as labeled antibodies which bind unmodified detection reagents or streptavidin modified with a detectable moiety for detecting biotin-modified detection reagents). As will be apparent to those of skill in the art, the reagents may also be bound to surfaces in forms other than an array.

The instructions relating to the use of the kit for carrying out the invention generally describe how the contents of the kit are used to carry out the methods of the invention. Instructions may include information such as sample requirements (e.g., form, pre-assay processing, and size), steps necessary to measure the AD diagnosis biomarkers, interpretation of results, etc.

Instructions supplied in the kits of the invention are typically written instructions on a label or package insert (e.g., a paper sheet included in the kit), but machine-readable instructions (e.g., instructions carried on a magnetic or optical storage disk) are also acceptable. In certain embodiments, machine-readable instructions comprise software for a programmable digital computer for comparing the measured levels obtained using the reagents included in the kit.

The following Examples are provided to illustrate the invention, but are not intended to limit the scope of the invention in any way.

### EXAMPLES

### Example 1

### AD VS. NDC Biomarker Differential Expression Test

The AD vs. NDC biomarker differential expression test was developed to determine or aid in the differentiation of AD from non-demented subjects. This test result may be combined with other clinical assessments, including cognitive assessment tools and clinical observation, in making a determination.

We examined more than 120 proteins detectable in plasma samples from 377 human subjects (Alzheimer's disease and non-demented controls) using Rules-Based Medicine (RBM) (Rules-Based Medicine, Inc., Austin, Tex.) to assay the samples on a Luminex platform (Luminex Corporation, Austin, Tex.). A signature panel of eleven (11) proteins was selected for the Alzheimer differentiation panel, and an algorithm capable of distinguishing AD samples from non-demented samples (NDC) was developed.

### 1) Sample Collection and Processing

At least 1 milliliter of whole blood was collected via venipuncture from the tested patient. The blood was collected into a collection tube containing the anticoagulant diaminoethanetetraacetic acid (EDTA). Following collection, the tube was inverted several times to mix the anticoagulant with the whole blood. The tube was centrifuged to separate the plasma from the cellular components or “pellet.” The supernatant (plasma) was decanted or removed by pipette and transferred into a sample storage tube or vessel. An aliquot of the plasma was tested at that time or frozen and stored for testing at a later time.

### 2) Assay Procedure

Plasma samples were assayed using the Luminex platform, a sandwich-format ELISA on a microsphere substrate, by a third party service provider (Rules Based Medicine).

In general, the Luminex platform is run as follows. An 8-point five-fold serial dilution series of antigen standard proteins in assay buffer is prepared, followed by preparation of dilution of plasma samples appropriate for detection of desired analytes.

Assay buffer in 150 μl is added to each filter plate, followed by a 5-minute incubation period. Capture antibody beads are added in 50 μL per well, and the plates are filtered and washed once with 150 μL/well Wash Buffer. Assay Buffer in 75 μL is then added to each well, followed by addition of samples or standards in 25 μL per well. Samples and standards are sealed and incubated for 30 minutes with gentle shaking at 500 rpm. The solution in the plates is removed, and the plates are washed three times with Buffer in 150 μL/well.

Detection antibody in 25 μL is added in each well, and samples and controls are sealed and incubated for 30 minutes with gentle shaking at 500 rpm. Solution is removed, and the plates are washed three times with Wash buffer in 150 μL per well. Streptavidin-PE conjugate in 50 μl/well is then added, and incubated for 30 minutes with gentle shaking at 500 rpm. The solution is then removed, and the samples and standards are washed 3 times with Wash Buffer with 150 μL per well.

Reading Buffer in 120 μL is added to each well. Samples and controls are sealed and incubated for 5 minutes with gentle shaking in 500 rpm, and are read in Luminex instrument. The fluorescent readings from each plasma samples are compared to the reading obtained from testing each standard curve and the plasma sample readings are converted to mass/volume concentration values.

Capture antibodies to each target protein are bound to a unique Luminex bead set which are collectively combined into between two and five multiplexes, or groupings of beads, co-optimized according to such parameters as cross-reactivity and sample dilution to yield maximum performance in terms of analytical sensitivity and dynamic concentration range.

### 3) Signature Panel Selection and Algorithm Development

To obtain a sample set that is representative of the general population, a total of 394 EDTA plasma AD and NDC samples obtained from eight of the top dementia clinical research centers from the US and Europe were used in the development of the signature and algorithm. Only those from centers with matched cases (e.g., matched for age and sex (based on clinical annotation data provided with the samples)) were used in the development phase of the signature panel selection and algorithm. Accordingly, approximately 377 samples from 8 centers were included in the further study. The AD samples selected for inclusion into the study were biased towards mild to moderate dementia based on the MMSE scores. In general, an “individual with mild dementia” is an individual who (a) has been diagnosed with dementia or has been given a diagnosis of probable dementia, and (b) has either been assessed with the Mini-Mental State Examination (MMSE) (referenced in Folstein et al., J. Psychiatr. Res 1975; 12:1289-198) and scored 22-27 or would achieve a score of 22-27 upon MMSE testing. In general, an “individual with moderate dementia” is an individual who (a) has been diagnosed with dementia or has been given a diagnosis of probable dementia, and (b) has either been assessed with the MMSE and scored 16-21 or would achieve a score of 16-21 upon MMSE testing.

A total of 150 protein biomarker concentrations were measured for each of the 377 samples. The list of biomarkers was reduced sequentially based on the principle of a decision tree. Sex-associated biomarkers and biomarkers associated with other, potentially confounding diseases or physiological conditions (for example, approximately 3 biomarkers only elevated in cancer patients) were removed as were biomarkers that were not detectable. The result was a list of 96 biomarkers. The data were normalized to the marker median of all samples and log transformed to normally distribute the data and then a number of different statistical tools were applied, including t-test, SAM, PAM, clutering, Elastic NET, Support Vector Machine, Logistic regression, Principle Components Analysis, etc., and biomarkers not significant in any of these analyses were eliminated from consideration.

To further reduce the number of biomarkers in the predictive signature, stepwise logistic regression was used and assessed using nested cross validation. At each step of this iterative process, logistic regression was performed on 90% of a given data set to derive a smaller but predictive set of biomarkers to optimized for that data set as assessed by internal cross validation and then measured for accuracy on the remaining 10% of the data set. Ten such combinations were performed resulting in 10 sets of predictive biomarkers. Only biomarkers that appeared in more than one of these 10 sets were kept for further analysis, resulting in a list reduced to 43 biomarkers. This was further reduced to 23 biomarkers by removing those biomarkers for which plasma samples were close to the limit of detection of the testing method used to generate the concentration data.

The list of biomarkers was further refined to a list of 11 biomarkers using non-normalized biomarker concentration data by testing various combinations of biomarkers in smaller and smaller sets, assessing performance with internal cross validation, to find the minimal number of biomarkers that provided the best estimated accuracy. This yielded the biomarker signature for AD versus NDC differentiation panel: ACE (angiotensin converting enzyme, CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand).

### 4) Scoring Algorithm and Result Reporting

Concentration of each protein AD diagnosis biomarker was entered into a scoring algorithm as shown in Table 2 for one sample.

TABLE 2
Example of Scoring Algorithm for One Sample

| | | Concen- | | Coefficient * |
|---|---|---|---|---|
| Variable | Coefficient | tration | Logged | Concentration |
| ACE (CD163) | 0.93727 | 163 | 5.0938 | 4.7743 |
| Alpha-1- | −1.86935 | 1.8 | 0.5906 | −1.1040 |
| Antitrypsin | | | | |
| Apo CIII | 0.43059 | 30.5 | 3.4179 | 1.4717 |
| Apo E | 0.30125 | 29.4 | 3.3812 | 1.0186 |
| Apo AI | 0.72352 | 0.28 | −1.2553 | −0.9082 |
| C Reactive | 0.26897 | 0.51 | −0.6636 | −0.1785 |
| Protein | | | | |
| Cortisol | −1.47408 | 119 | 4.7792 | −7.0449 |
| FGF-4 | −0.33876 | 301 | 5.7071 | −1.9334 |
| MCP-3 | −0.44652 | 18 | 2.8906 | −1.2907 |
| MMP-9 | −0.39390 | 174 | 5.1591 | −2.0322 |
| TRAIL R3 | 0.71845 | 14 | 2.6394 | 1.8963 |
| (Intercept) | 4.80238 | 1 | 1 | 4.8024 |

| | | |
|---|---|---|
| | Logged Score | −0.5286 |
| | Score | 0.3708 |
| Cutoff is <0.5 | Prediction | AD |

The coefficients for each biomarker were indications of the relative weight of the biomarkers in making the prediction. The coefficients were developed to maximize the separation of the 2 sets of samples (NDC vs. AD) and the intercept was developed by scaling to adjust the readout to a range of 0 to 1.

The calculated formulaic result or score (inverse logit) was based on the formula:

Score=(e^{(sum)})/(1+e^{(sum)}),

and the sum was calculated in accordance with the formula:

Sum=Intercept+ΣCoefficient(AD diagnosis biomarkerᵢ)*ln [AD diagnosis biomarkerᵢ+0.005], i=1 to N,N=number of AD diagnosis biomarker; N=number of AD diagnosis biomarkers used, and [AD diagnosis biomarker]=Actual concentration value of each AD diagnosis biomarker (*=X=multiplication)

The calculated value of score was compared to a cutoff score, and a prediction of AD versus NDC was made. The cutoff score was determined by reviewing the distribution of calls and comparing them with the true diagnosis, and was selected to provide maximum accuracy in calling either AD or NDC. A balance between sensitivity and specificity was taken into account when determining these values. The closer a result or score was to zero (in the case of AD) or one (in the case of NDC), the greater the confidence or probability that the prediction was more accurate. For example, when the cutoff score was 0.5, a score lower than 0.5 indicates that a test subject has AD, and a score higher or equal to 0.5 indicates that a test subject is non-demented.

The cutoff score can be varied to achieve optimized sensitivity or specificity in the assay. The cutoff can be adjusted using the ROC curve with a fitted equation if there were a cost/benefit rationale for favoring one parameter over the other.

The parameters of the algorithm, including coefficients and intercept, can be optimized over time when additional samples are tested.

Every possible combination of the 11 biomarkers in the signature was used to generate a separate logistic regression algorithm. Each of these algorithms was applied to the entire set of samples to generate scores and predict calls for each sample using each of 10 cutoffs (from 0.1 to 1.0 in 0.1-steps). The percentage of samples predicted correctly (Accuracy) was measured for each of the subset algorithms at each cutoff (called a model). 498 models with 65% Accuracy or higher were deemed to be of potential utility based on the observation that a genomics-based test approved by the FDA has a similar level of performance deemed to be useful as an aid to diagnosis (Xdx test has 67% AUC). These 498 models (with coefficients) are shown in Table 3.

Accordingly, in various embodiments of the invention for use with any of the methods, kits, surfaces, or computer-readable storage formats or mediums described herein, the set of AD diagnosis biomarkers may comprise any set shown in Table 3.

TABLE 3
Scoring Algorithms With Greater Than 65% Accuracy

| | | | % | | | | | | | | | | | | |
|---|---|---|---|---|---|---|---|---|---|---|---|---|---|---|---|
| | | | Ac- | | Alpha- | | | | C Re- | | | | | | |
| | Cut- | No of | cu- | CE/ | 1-Anti- | Apo | | | active | | | | | TRAIL | In- |
| Model | off | Markers | racy | ACD143 | trypsin | CIII | Apo E | Apo A1 | Protein | Cortisol | FGF-4 | MCP-3 | MMP-9 | R3 | tercept |
| 2047 | 0.3 | 11 | 66 | 0.9373 | −1.8693 | 0.4306 | 0.3012 | 0.7235 | 0.2690 | −1.4741 | −0.3388 | −0.4465 | −0.3939 | 0.7185 | 4.8024 |
| 2047 | 0.4 | 11 | 66 | 0.9373 | −1.8693 | 0.4306 | 0.3012 | 0.7235 | 0.2690 | −1.4741 | −0.3388 | −0.4465 | −0.3939 | 0.7185 | 4.8024 |
| 2047 | 0.5 | 11 | 65.5 | 0.9373 | −1.8693 | 0.4306 | 0.3012 | 0.7235 | 0.2690 | −1.4741 | −0.3388 | −0.4465 | −0.3939 | 0.7185 | 4.8024 |
| 2037 | 0.4 | 10 | 65 | 0.9976 | −1.8998 | 0.4167 | 0.3095 | 0.7133 | 0.2705 | −1.4052 | −0.2824 | −0.4208 | | 0.5927 | 2.2696 |
| 2044 | 0.5 | 10 | 65.2 | | −1.7213 | 0.3160 | 0.3525 | 0.8061 | 0.2460 | −1.4622 | −0.2531 | −0.5084 | −0.4295 | 0.8570 | 9.2714 |
| 2036 | 0.3 | 10 | 65.5 | 1.0132 | −1.6744 | 0.2656 | 0.3759 | 0.7866 | 0.2595 | −1.3986 | −0.4666 | | −0.3766 | 0.5501 | 4.0834 |
| 2045 | 0.4 | 10 | 65.5 | 0.9848 | −1.8369 | 0.4369 | | 0.6270 | 0.3055 | −1.4439 | −0.2692 | −0.5128 | −0.3940 | 0.6912 | 5.0848 |
| 2040 | 0.5 | 10 | 65.5 | 0.8490 | −1.7389 | | 0.3031 | 0.9592 | 0.2890 | −1.4354 | −0.4133 | −0.3349 | −0.3871 | 0.6437 | 7.3153 |
| 2040 | 0.6 | 10 | 65.5 | 0.8490 | −1.7389 | | 0.3031 | 0.9592 | 0.2890 | −1.4354 | −0.4133 | −0.3349 | −0.3871 | 0.6437 | 7.3153 |
| 2045 | 0.5 | 10 | 65.5 | 0.9848 | −1.8369 | 0.4369 | | 0.6270 | 0.3055 | −1.4439 | −0.2692 | −0.5128 | −0.3940 | 0.6912 | 5.0848 |
| 2043 | 0.3 | 10 | 65.7 | 0.9817 | −1.5120 | 0.6056 | 0.2448 | | 0.2245 | −1.4054 | −0.3291 | −0.4761 | −0.3902 | 0.7521 | 2.5038 |
| 2044 | 0.4 | 10 | 66 | | −1.7213 | 0.3160 | 0.3525 | 0.8061 | 0.2460 | −1.4622 | −0.2531 | −0.5084 | −0.4295 | 0.8570 | 9.2714 |
| 2043 | 0.4 | 10 | 66 | 0.9817 | −1.5120 | 0.6056 | 0.2448 | | 0.2245 | −1.4054 | −0.3291 | −0.4761 | −0.3902 | 0.7521 | 2.5038 |
| 2038 | 0.4 | 10 | 66.2 | 0.8841 | −1.5290 | 0.4889 | 0.3801 | 0.5474 | | −1.5515 | −0.3873 | −0.4275 | −0.3929 | 0.8422 | 4.4371 |
| 2036 | 0.4 | 10 | 66.2 | 1.0132 | −1.6744 | 0.2656 | 0.3759 | 0.7866 | 0.2595 | −1.3986 | −0.4666 | | −0.3766 | 0.5501 | 4.0834 |
| 2046 | 0.4 | 10 | 66.5 | 0.8404 | −1.8975 | 0.5397 | 0.2221 | 0.7045 | 0.2963 | −1.4552 | | −0.5743 | −0.3539 | 0.7030 | 3.3675 |
| 2037 | 0.5 | 10 | 66.5 | 0.9976 | −1.8998 | 0.4167 | 0.3095 | 0.7133 | 0.2705 | −1.4052 | −0.2824 | −0.4208 | | 0.5927 | 2.2696 |
| 2038 | 0.5 | 10 | 66.5 | 0.8841 | −1.5290 | 0.4889 | 0.3801 | 0.5474 | | −1.5515 | −0.3873 | −0.4275 | −0.3929 | 0.8422 | 4.4371 |
| 2046 | 0.5 | 10 | 66.8 | 0.8404 | −1.8975 | 0.5397 | 0.2221 | 0.7045 | 0.2963 | −1.4552 | | −0.5743 | −0.3539 | 0.7030 | 3.3675 |
| 2040 | 0.4 | 10 | 67 | 0.8490 | −1.7389 | | 0.3031 | 0.9592 | 0.2890 | −1.4354 | −0.4133 | −0.3349 | −0.3871 | 0.6437 | 7.3153 |
| 2039 | 0.5 | 10 | 67.8 | 0.8428 | | 0.3238 | 0.2625 | 0.3055 | 0.1691 | −1.7191 | −0.3540 | −0.3326 | −0.4054 | 0.5159 | 5.4146 |
| 2042 | 0.4 | 10 | 68.3 | 1.0405 | −1.6320 | 0.3556 | 0.2800 | 0.7722 | 0.3098 | −1.3591 | −0.3272 | −0.3347 | −0.3352 | | 5.0279 |
| 2039 | 0.4 | 10 | 68.3 | 0.8428 | | 0.3238 | 0.2625 | 0.3055 | 0.1691 | −1.7191 | −0.3540 | −0.3326 | −0.4054 | 0.5159 | 5.4146 |
| 2014 | 0.4 | 9 | 65 | 1.0064 | −2.0731 | 0.3081 | 0.2524 | 0.6373 | 0.3372 | | −0.3180 | −0.2938 | −0.3075 | | −0.8751 |
| 2017 | 0.6 | 9 | 65 | 0.6934 | −1.7405 | | 0.2049 | 1.0230 | 0.3314 | −1.4034 | | −0.4643 | −0.3353 | 0.5950 | 6.3473 |
| 1983 | 0.4 | 9 | 65 | 1.0188 | −1.5214 | 0.5919 | | | 0.2591 | −1.3875 | −0.2714 | −0.5279 | −0.3899 | 0.7242 | 2.9718 |
| 2003 | 0.4 | 9 | 65 | 1.0479 | −1.8573 | 0.4249 | | 0.6119 | 0.3060 | −1.3711 | −0.2110 | −0.4864 | | 0.5644 | 2.5179 |
| 1985 | 0.5 | 9 | 65 | 0.9383 | −1.4234 | 0.5087 | | 0.3859 | | −1.5226 | −0.3046 | −0.5083 | −0.3912 | 0.8219 | 4.7064 |
| 1982 | 0.5 | 9 | 65 | 0.8874 | −1.5386 | 0.7098 | 0.1689 | | 0.2505 | −1.3863 | | −0.6009 | −0.3533 | 0.7386 | 1.1415 |
| 2002 | 0.4 | 9 | 65.2 | 0.9117 | −1.9306 | 0.5117 | 0.2408 | 0.7019 | 0.2941 | −1.3931 | | −0.5314 | | 0.5923 | 1.2589 |
| 2026 | 0.5 | 9 | 65.2 | | −1.7574 | 0.4078 | 0.2907 | 0.7850 | 0.2676 | −1.4472 | | −0.6002 | −0.3961 | 0.8349 | 7.8017 |
| 1996 | 0.4 | 9 | 65.2 | 0.8926 | −1.6373 | 0.3600 | 0.2902 | 0.7931 | 0.2962 | −1.3292 | | | −0.3080 | 0.4444 | 1.5714 |
| 2017 | 0.5 | 9 | 65.2 | 0.6934 | −1.7405 | | 0.2049 | 1.0230 | 0.3314 | −1.4034 | | −0.4643 | −0.3353 | 0.5950 | 6.3473 |
| 2030 | 0.5 | 9 | 65.2 | | | 0.2279 | 0.3104 | 0.4031 | 0.1556 | −1.6856 | −0.2774 | −0.3990 | −0.4382 | 0.6563 | 9.4139 |
| 1990 | 0.6 | 9 | 65.2 | 0.8212 | | 0.3778 | 0.3191 | 0.2379 | | −1.7435 | −0.3830 | −0.3370 | −0.4050 | 0.6234 | 5.1006 |
| 2001 | 0.3 | 9 | 65.5 | 1.0869 | −1.5264 | 0.2416 | 0.3436 | 0.8143 | 0.2958 | −1.3206 | −0.4303 | | −0.3341 | | 4.3819 |
| 2035 | 0.4 | 9 | 65.5 | | −1.6381 | | 0.3471 | 0.9809 | 0.2631 | −1.4357 | −0.3143 | −0.4191 | −0.4226 | 0.7920 | 10.8644 |
| 2035 | 0.5 | 9 | 65.5 | | −1.6381 | | 0.3471 | 0.9809 | 0.2631 | −1.4357 | −0.3143 | −0.4191 | −0.4226 | 0.7920 | 10.8644 |
| 1994 | 0.5 | 9 | 65.5 | 1.0063 | −1.1955 | 0.4134 | 0.3637 | 0.5738 | | −1.4255 | −0.3829 | −0.2966 | −0.3243 | | 4.6436 |
| 2029 | 0.5 | 9 | 65.5 | | −1.4257 | 0.3766 | 0.4206 | 0.6394 | | −1.5321 | −0.3005 | −0.4931 | −0.4255 | 0.9704 | 8.6872 |
| 2003 | 0.5 | 9 | 65.5 | 1.0479 | −1.8573 | 0.4249 | | 0.6119 | 0.3060 | −1.3711 | −0.2110 | −0.4864 | | 0.5644 | 2.5179 |
| 2024 | 0.4 | 9 | 65.5 | 0.9121 | −1.7776 | | 0.3120 | 0.9434 | 0.2900 | −1.3760 | −0.3579 | −0.3133 | | 0.5247 | 4.7822 |
| 2029 | 0.4 | 9 | 65.7 | | −1.4257 | 0.3766 | 0.4206 | 0.6394 | | −1.5321 | −0.3005 | −0.4931 | −0.4255 | 0.9704 | 8.6872 |
| 1994 | 0.4 | 9 | 65.7 | 1.0063 | −1.1955 | 0.4134 | 0.3637 | 0.5738 | | −1.4255 | −0.3829 | −0.2966 | −0.3243 | | 4.6436 |
| 2018 | 0.4 | 9 | 65.7 | 0.8933 | −1.7039 | | | 0.8651 | 0.3266 | −1.4019 | −0.3452 | −0.4001 | −0.3886 | 0.6129 | 7.6526 |
| 2008 | 0.4 | 9 | 65.7 | 1.0721 | −1.7188 | 0.2616 | 0.3795 | 0.7780 | 0.2639 | −1.3362 | −0.4068 | | | 0.4432 | 1.6729 |
| 2033 | 0.5 | 9 | 65.7 | | −1.7570 | 0.2963 | 0.3677 | 0.8077 | 0.2451 | −1.3824 | −0.1855 | −0.4898 | | 0.7360 | 6.7887 |
| 2006 | 0.4 | 9 | 65.7 | 0.9125 | | 0.3123 | 0.2625 | 0.2879 | 0.1699 | −1.6433 | −0.3008 | −0.3085 | | 0.3793 | 2.7824 |
| 2022 | 0.5 | 9 | 65.7 | 0.9573 | −1.5507 | | 0.2819 | 0.9662 | 0.3239 | −1.3327 | −0.3896 | −0.2533 | −0.3357 | | 7.1211 |
| 2002 | 0.5 | 9 | 65.7 | 0.9117 | −1.9306 | 0.5117 | 0.2408 | 0.7019 | 0.2941 | −1.3931 | | −0.5314 | | 0.5923 | 1.2589 |
| 2004 | 0.5 | 9 | 65.7 | 1.0465 | −1.5495 | 0.5882 | 0.2517 | | 0.2259 | −1.3293 | −0.2754 | −0.4519 | | 0.6248 | −0.0052 |
| 2006 | 0.5 | 9 | 65.7 | 0.9125 | | 0.3123 | 0.2625 | 0.2879 | 0.1699 | −1.6433 | −0.3008 | −0.3085 | | 0.3793 | 2.7824 |
| 2009 | 0.5 | 9 | 66 | 0.8295 | −2.3176 | 0.4759 | 0.1924 | 0.5641 | 0.3340 | | | −0.4967 | −0.3088 | 0.5410 | −2.8069 |
| 1992 | 0.4 | 9 | 66 | 1.0800 | −1.6023 | 0.3624 | | 0.6768 | 0.3411 | −1.3377 | −0.2633 | −0.4006 | −0.3376 | | 5.3055 |
| 1990 | 0.4 | 9 | 66 | 0.8212 | | 0.3778 | 0.3191 | 0.2379 | | −1.7435 | −0.3830 | −0.3370 | −0.4050 | 0.6234 | 5.1006 |
| 2023 | 0.4 | 9 | 66 | 0.9397 | −1.6199 | | 0.3641 | 0.9363 | 0.2755 | −1.3852 | −0.4956 | | −0.3748 | 0.5298 | 5.9178 |
| 1981 | 0.5 | 9 | 66 | 0.8951 | −1.8654 | 0.5283 | | 0.6315 | 0.3203 | −1.4370 | | −0.6060 | −0.3601 | 0.6842 | 3.8095 |
| 1985 | 0.4 | 9 | 66.2 | 0.9383 | −1.4234 | 0.5087 | | 0.3859 | | −1.5226 | −0.3046 | −0.5083 | −0.3912 | 0.8219 | 4.7064 |
| 2022 | 0.4 | 9 | 66.2 | 0.9573 | −1.5507 | | 0.2819 | 0.9662 | 0.3239 | −1.3327 | −0.3896 | −0.2533 | −0.3357 | | 7.1211 |
| 1986 | 0.5 | 9 | 66.2 | 0.9270 | −1.2980 | 0.6190 | 0.3246 | | | −1.4813 | −0.3715 | −0.4534 | −0.3899 | 0.8531 | 2.6357 |
| 1998 | 0.5 | 9 | 66.2 | 1.0730 | −1.2747 | 0.4422 | 0.3189 | | 0.2101 | −1.3174 | −0.4699 | | −0.3734 | 0.5773 | 1.5351 |
| 1993 | 0.4 | 9 | 66.5 | 1.1014 | −1.2409 | 0.5378 | 0.2157 | | 0.2646 | −1.2827 | −0.3178 | −0.3618 | −0.3267 | | 2.5731 |
| 2020 | 0.4 | 9 | 66.5 | 0.7785 | −1.3559 | | 0.3899 | 0.8039 | | −1.5200 | −0.4773 | −0.3008 | −0.3847 | 0.7689 | 7.3278 |
| 1982 | 0.4 | 9 | 66.5 | 0.8874 | −1.5386 | 0.7098 | 0.1689 | | 0.2505 | −1.3863 | | −0.6009 | −0.3533 | 0.7386 | 1.1415 |
| 2028 | 0.5 | 9 | 66.5 | | −1.3152 | 0.5094 | 0.2956 | | 0.1948 | −1.3784 | −0.2368 | −0.5512 | −0.4300 | 0.9083 | 6.8933 |
| 1999 | 0.5 | 9 | 66.5 | 0.9658 | −1.3647 | 0.3321 | 0.4462 | 0.6121 | | −1.4767 | −0.5076 | | −0.3791 | 0.6836 | 3.7209 |
| 1995 | 0.5 | 9 | 66.5 | 0.9304 | | 0.2825 | 0.2458 | 0.3781 | 0.2090 | −1.6102 | −0.3422 | −0.2630 | −0.3606 | | 5.5056 |
| 2005 | 0.5 | 9 | 66.5 | 0.9450 | −1.5638 | 0.4740 | 0.3853 | 0.5342 | | −1.4765 | −0.3326 | −0.4065 | | 0.7186 | 1.9138 |
| 2005 | 0.4 | 9 | 66.8 | 0.9450 | −1.5638 | 0.4740 | 0.3853 | 0.5342 | | −1.4765 | −0.3326 | −0.4065 | | 0.7186 | 1.9138 |
| 1984 | 0.5 | 9 | 66.8 | 0.7623 | −1.5161 | 0.6234 | 0.2970 | 0.4957 | | −1.5378 | | −0.5736 | −0.3489 | 0.8382 | 2.7388 |
| 2000 | 0.4 | 9 | 66.8 | 0.9107 | | 0.2059 | 0.3196 | 0.3859 | 0.1719 | −1.6397 | −0.4543 | | −0.3927 | 0.4061 | 4.8257 |
| 1984 | 0.4 | 9 | 66.8 | 0.7623 | −1.5161 | 0.6234 | 0.2970 | 0.4957 | | −1.5378 | | −0.5736 | −0.3489 | 0.8382 | 2.7388 |
| 1990 | 0.5 | 9 | 66.8 | 0.8212 | | 0.3778 | 0.3191 | 0.2379 | | −1.7435 | −0.3830 | −0.3370 | −0.4050 | 0.6234 | 5.1006 |
| 2032 | 0.4 | 9 | 67 | | −1.5046 | 0.1142 | 0.4349 | 0.8993 | 0.2376 | −1.3636 | −0.3913 | | −0.4174 | 0.6849 | 8.8682 |
| 1988 | 0.4 | 9 | 67 | 0.8881 | | 0.3306 | | 0.2288 | 0.2025 | −1.6887 | −0.2913 | −0.3913 | −0.4030 | 0.4909 | 5.6274 |
| 2019 | 0.4 | 9 | 67 | 0.8675 | −1.1408 | | 0.2216 | | 0.2342 | −1.3105 | −0.4517 | −0.3111 | −0.3781 | 0.6495 | 5.4023 |
| 1995 | 0.4 | 9 | 67 | 0.9304 | | 0.2825 | 0.2458 | 0.3781 | 0.2090 | −1.6102 | −0.3422 | −0.2630 | −0.3606 | | 5.5056 |
| 1989 | 0.5 | 9 | 67 | 0.8714 | | 0.4167 | 0.2394 | | 0.1572 | −1.6565 | −0.3463 | −0.3564 | −0.4034 | 0.5493 | 4.2326 |
| 2030 | 0.4 | 9 | 67.3 | | | 0.2279 | 0.3104 | 0.4031 | 0.1556 | −1.6856 | −0.2774 | −0.3990 | −0.4382 | 0.6563 | 9.4139 |
| 1987 | 0.5 | 9 | 67.3 | 0.7480 | | 0.4413 | 0.1780 | 0.2653 | 0.1936 | −1.7100 | | −0.4681 | −0.3679 | 0.4923 | 3.9175 |
| 1988 | 0.5 | 9 | 67.3 | 0.8881 | | 0.3306 | | 0.2288 | 0.2025 | −1.6887 | −0.2913 | −0.3913 | −0.4030 | 0.4909 | 5.6274 |
| 2000 | 0.5 | 9 | 67.3 | 0.9107 | | 0.2059 | 0.3196 | 0.3859 | 0.1719 | −1.6397 | −0.4543 | | −0.3927 | 0.4061 | 4.8257 |
| 2001 | 0.4 | 9 | 67.5 | 1.0869 | −1.5264 | 0.2416 | 0.3436 | 0.8143 | 0.2958 | −1.3206 | −0.4303 | | −0.3341 | | 4.3819 |
| 1987 | 0.4 | 9 | 67.5 | 0.7480 | | 0.4413 | 0.1780 | 0.2653 | 0.1936 | −1.7100 | | −0.4681 | −0.3679 | 0.4923 | 3.9175 |
| 2021 | 0.5 | 9 | 67.5 | 0.7812 | | | 0.2653 | 0.5105 | 0.1905 | −1.6805 | −0.4123 | −0.2547 | −0.4010 | 0.4741 | 7.3355 |
| 1991 | 0.4 | 9 | 67.8 | 0.9450 | −1.6625 | 0.4596 | 0.2042 | 0.7560 | 0.3358 | −1.3435 | | −0.4591 | −0.2997 | | 3.6556 |
| 2020 | 0.5 | 9 | 67.8 | 0.7785 | −1.3559 | | 0.3899 | 0.8039 | | −1.5200 | −0.4773 | −0.3008 | −0.3847 | 0.7689 | 7.3278 |
| 1989 | 0.4 | 9 | 68.3 | 0.8714 | | 0.4167 | 0.2394 | | 0.1572 | −1.6565 | −0.3463 | −0.3564 | −0.4034 | 0.5493 | 4.2326 |
| 2021 | 0.4 | 9 | 69 | 0.7812 | | | 0.2653 | 0.5105 | 0.1905 | −1.6805 | −0.4123 | −0.2547 | −0.4010 | 0.4741 | 7.3355 |
| 1907 | 0.4 | 8 | 65 | | −1.4865 | | 0.4269 | 0.9634 | 0.2453 | −1.3600 | −0.4068 | | −0.4154 | 0.6715 | 9.5460 |
| 1926 | 0.4 | 8 | 65 | 0.9389 | −1.9972 | | 0.2537 | 0.8097 | 0.3506 | | −0.3753 | −0.2237 | −0.3112 | | 1.0569 |
| 1871 | 0.5 | 8 | 65 | 0.9641 | −1.6180 | 0.3783 | 0.3434 | 0.3933 | | | −0.3777 | −0.2528 | −0.2915 | | −1.6732 |
| 1891 | 0.4 | 8 | 65 | | | 0.2810 | 0.3603 | 0.3372 | | −1.7089 | −0.3050 | −0.4036 | −0.4363 | 0.7561 | 9.0156 |
| 1910 | 0.5 | 8 | 65 | 0.8947 | −2.1367 | | | 0.7172 | 0.3573 | | −0.3354 | −0.3427 | −0.3497 | 0.4688 | 1.2950 |
| 1958 | 0.4 | 8 | 65 | 1.1294 | −1.5873 | 0.2435 | 0.3526 | 0.7975 | 0.2931 | −1.2800 | −0.3844 | | | | 2.1397 |
| 1853 | 0.4 | 8 | 65 | 0.9610 | −1.5179 | 0.3345 | 0.2702 | 0.8175 | 0.3246 | −1.2707 | | | −0.2804 | | 1.9909 |
| 1901 | 0.5 | 8 | 65 | 0.7447 | −1.7120 | | | 0.9474 | 0.3534 | −1.3854 | | −0.4964 | −0.3422 | 0.5786 | 6.7028 |
| 1909 | 0.5 | 8 | 65 | | −1.0204 | | 0.2644 | | 0.2063 | −1.3014 | −0.3484 | −0.4020 | −0.4164 | 0.8097 | 8.9477 |
| 1919 | 0.5 | 8 | 65 | | −1.3038 | | 0.4211 | 0.8366 | | −1.5097 | −0.3799 | −0.3856 | −0.4170 | 0.9016 | 10.5943 |
| 1925 | 0.6 | 8 | 65 | | −1.3907 | | 0.3287 | 1.0039 | 0.3052 | −1.3113 | −0.2706 | −0.3381 | −0.3664 | | 11.2892 |
| 1836 | 0.5 | 8 | 65 | 1.0428 | −1.1910 | 0.3189 | | 0.4292 | | −1.4253 | −0.4390 | | −0.3761 | 0.6235 | 3.9534 |
| 1943 | 0.5 | 8 | 65 | | −1.5509 | 0.1030 | 0.4469 | 0.9029 | 0.2396 | −1.2841 | −0.3229 | | | 0.5775 | 6.4531 |
| 1819 | 0.5 | 8 | 65 | 0.9531 | −1.2209 | 0.5419 | 0.2319 | | 0.2444 | −1.2404 | | | −0.3072 | 0.4736 | −1.0654 |
| 1904 | 0.5 | 8 | 65 | 0.9568 | −1.0458 | | 0.2793 | | 0.2234 | −1.2655 | −0.5303 | | −0.3691 | 0.5469 | 4.1389 |
| 1839 | 0.5 | 8 | 65 | 0.9664 | −1.2628 | 0.6025 | | | | −1.4729 | −0.3011 | −0.5188 | −0.3886 | 0.8320 | 3.3265 |
| 1841 | 0.6 | 8 | 65 | 0.8079 | −1.3008 | 0.7392 | 0.2488 | | | −1.4714 | | −0.5937 | −0.3490 | 0.8498 | 1.1350 |
| 1922 | 0.6 | 8 | 65 | 1.0172 | −1.4850 | | 0.3331 | 0.9492 | 0.3091 | −1.3084 | −0.4577 | | −0.3342 | | 6.0399 |
| 1851 | 0.4 | 8 | 65.2 | 1.1436 | −1.4533 | 0.2201 | | 0.7007 | 0.3316 | −1.2835 | −0.3770 | | −0.3368 | | 4.6172 |
| 1900 | 0.4 | 8 | 65.2 | 1.0130 | −1.5366 | | | 0.8098 | 0.3173 | −1.3333 | −0.4328 | | −0.3733 | 0.4633 | 6.0323 |
| 1959 | 0.4 | 8 | 65.2 | 0.9952 | −1.7189 | 0.4494 | 0.2222 | 0.7436 | 0.3288 | −1.3072 | | −0.4411 | | | 1.7777 |
| 1847 | 0.5 | 8 | 65.2 | 0.9264 | −1.7609 | 0.3104 | 0.4092 | 0.4142 | | | −0.4798 | | −0.3346 | 0.5573 | −2.6297 |
| 1839 | 0.4 | 8 | 65.2 | 0.9664 | −1.2628 | 0.6025 | | | | −1.4729 | −0.3011 | −0.5188 | −0.3886 | 0.8320 | 3.3265 |
| 1927 | 0.4 | 8 | 65.2 | 0.9060 | −1.0819 | | 0.3711 | 0.7910 | | −1.4054 | −0.4593 | −0.2015 | −0.3244 | | 7.1266 |
| 1952 | 0.4 | 8 | 65.2 | 1.0253 | −1.4057 | 0.3274 | 0.4485 | 0.5973 | | −1.4092 | −0.4492 | | | 0.5753 | 1.2831 |
| 1916 | 0.4 | 8 | 65.2 | 0.8679 | −1.2722 | | 0.4383 | 0.7899 | | −1.4696 | −0.5480 | | −0.3763 | 0.6661 | 6.0480 |
| 1897 | 0.5 | 8 | 65.2 | | | 0.1606 | 0.2949 | 0.5183 | 0.2079 | −1.5424 | −0.2537 | −0.3227 | −0.3879 | | 10.1562 |
| 1952 | 0.5 | 8 | 65.2 | 1.0253 | −1.4057 | 0.3274 | 0.4485 | 0.5973 | | −1.4092 | −0.4492 | | | 0.5753 | 1.2831 |
| 1954 | 0.4 | 8 | 65.2 | 0.9902 | −1.3379 | 0.6000 | 0.3299 | | | −1.4036 | −0.3188 | −0.4318 | | 0.7288 | 0.1510 |
| 1924 | 0.5 | 8 | 65.2 | 0.9864 | −0.9565 | | 0.1951 | | 0.2694 | −1.2055 | −0.4282 | −0.2318 | −0.3246 | | 5.1681 |
| 1899 | 0.5 | 8 | 65.2 | 0.9296 | | 0.3414 | 0.3134 | 0.3080 | | −1.6126 | −0.3782 | −0.2520 | −0.3490 | | 5.1277 |
| 1903 | 0.5 | 8 | 65.2 | 0.9002 | −1.1506 | | | | 0.2653 | −1.2931 | −0.3965 | −0.3613 | −0.3792 | 0.6232 | 5.7766 |
| 1970 | 0.5 | 8 | 65.2 | 0.8901 | | 0.3655 | 0.3176 | 0.2162 | | −1.6648 | −0.3302 | −0.3146 | | 0.4884 | 2.4690 |
| 1843 | 0.4 | 8 | 65.5 | | −1.2323 | 0.1797 | 0.4970 | 0.7347 | | −1.4339 | −0.4323 | | −0.4159 | 0.8048 | 8.3133 |
| 1872 | 0.4 | 8 | 65.5 | 0.9772 | | 0.1874 | | 0.3072 | 0.2143 | −1.5836 | −0.3996 | | −0.3879 | 0.3494 | 5.0174 |
| 1837 | 0.4 | 8 | 65.5 | 0.8313 | −1.4285 | 0.6179 | | 0.3713 | | −1.5193 | | −0.6159 | −0.3549 | 0.8223 | 3.2451 |
| 1881 | 0.4 | 8 | 65.5 | | | 0.3493 | 0.2836 | | 0.1387 | −1.6011 | −0.2635 | −0.4360 | −0.4379 | 0.7106 | 8.0093 |
| 1841 | 0.4 | 8 | 65.5 | 0.8079 | −1.3008 | 0.7392 | 0.2488 | | | −1.4714 | | −0.5937 | −0.3490 | 0.8498 | 1.1350 |
| 1940 | 0.4 | 8 | 65.5 | 1.1361 | −1.3238 | 0.4357 | 0.3213 | | 0.2137 | −1.2471 | −0.4120 | | | 0.4663 | −0.8677 |
| 1975 | 0.4 | 8 | 65.5 | 0.9352 | −1.1959 | | 0.2284 | | 0.2348 | −1.2444 | −0.3993 | −0.2949 | | 0.5311 | 2.9310 |
| 1890 | 0.4 | 8 | 65.5 | 0.8448 | | 0.4479 | 0.2973 | | | −1.6914 | −0.3749 | −0.3552 | −0.4031 | 0.6435 | 4.1756 |
| 1979 | 0.5 | 8 | 65.5 | 0.9984 | −1.6115 | | 0.2925 | 0.9485 | 0.3198 | −1.2979 | −0.3457 | −0.2496 | | | 4.9036 |
| 1867 | 0.5 | 8 | 65.5 | 1.0542 | −1.1246 | 0.3121 | 0.4151 | 0.6181 | | −1.3895 | −0.4708 | | −0.3253 | | 4.0601 |
| 1966 | 0.4 | 8 | 65.5 | 0.8244 | | 0.4140 | 0.1893 | 0.2554 | 0.1911 | −1.6394 | | −0.4260 | | 0.3710 | 1.6947 |
| 1974 | 0.5 | 8 | 65.5 | 0.7691 | −1.7827 | | 0.2232 | 1.0060 | 0.3281 | −1.3532 | | −0.4298 | | 0.4978 | 4.2211 |
| 1967 | 0.4 | 8 | 65.5 | 0.9402 | | 0.3991 | 0.2403 | | 0.1580 | −1.5815 | −0.2938 | −0.3313 | | 0.4104 | 1.6699 |
| 1975 | 0.5 | 8 | 65.5 | 0.9352 | −1.1959 | | 0.2284 | | 0.2348 | −1.2444 | −0.3993 | −0.2949 | | 0.5311 | 2.9310 |
| 1915 | 0.5 | 8 | 65.5 | 0.8263 | −1.2351 | | | 0.6460 | | −1.4871 | −0.3979 | −0.3775 | −0.3854 | 0.7414 | 7.7529 |
| 1939 | 0.5 | 8 | 65.5 | 1.0855 | −1.5522 | 0.5748 | | | 0.2602 | −1.3086 | −0.2162 | −0.5030 | | 0.5963 | 0.4506 |
| 1966 | 0.5 | 8 | 65.5 | 0.8244 | | 0.4140 | 0.1893 | 0.2554 | 0.1911 | −1.6394 | | −0.4260 | | 0.3710 | 1.6947 |
| 1879 | 0.4 | 8 | 65.7 | | | 0.0734 | 0.3809 | 0.5172 | 0.1592 | −1.5818 | −0.3896 | | −0.4279 | 0.5399 | 9.0848 |
| 1924 | 0.4 | 8 | 65.7 | 0.9864 | −0.9565 | | 0.1951 | | 0.2694 | −1.2055 | −0.4282 | −0.2318 | −0.3246 | | 5.1681 |
| 1915 | 0.4 | 8 | 65.7 | 0.8263 | −1.2351 | | | 0.6460 | | −1.4871 | −0.3979 | −0.3775 | −0.3854 | 0.7414 | 7.7529 |
| 1932 | 0.4 | 8 | 65.7 | | | | 0.3079 | 0.5460 | 0.1719 | −1.6612 | −0.3227 | −0.3391 | −0.4334 | 0.6187 | 10.5969 |
| 1927 | 0.5 | 8 | 65.7 | 0.9060 | −1.0819 | | 0.3711 | 0.7910 | | −1.4054 | −0.4593 | −0.2015 | −0.3244 | | 7.1266 |
| 1932 | 0.5 | 8 | 65.7 | | | | 0.3079 | 0.5460 | 0.1719 | −1.6612 | −0.3227 | −0.3391 | −0.4334 | 0.6187 | 10.5969 |
| 1923 | 0.4 | 8 | 65.7 | 0.8035 | −1.5664 | | 0.1907 | 1.0270 | 0.3626 | −1.3089 | | −0.3817 | −0.2924 | | 6.2186 |
| 1960 | 0.4 | 8 | 65.7 | 1.1448 | −1.3103 | 0.5347 | 0.2258 | | 0.2607 | −1.2386 | −0.2754 | −0.3602 | | | 0.4044 |
| 1902 | 0.4 | 8 | 65.7 | 0.7816 | −1.5644 | | 0.2694 | 1.0097 | 0.3219 | −1.3121 | | | −0.3012 | 0.4081 | 3.9375 |
| 1837 | 0.5 | 8 | 65.7 | 0.8313 | −1.4285 | 0.6179 | | 0.3713 | | −1.5193 | | −0.6159 | −0.3549 | 0.8223 | 3.2451 |
| 1955 | 0.5 | 8 | 65.7 | | −1.4706 | 0.3573 | 0.4326 | 0.6373 | | −1.4476 | −0.2349 | −0.4790 | | 0.8527 | 6.2308 |
| 1918 | 0.5 | 8 | 65.7 | 0.8072 | −0.9152 | | 0.3033 | | | −1.3900 | −0.4976 | −0.2874 | −0.3764 | 0.7537 | 5.6304 |
| 1963 | 0.5 | 8 | 65.7 | 1.0494 | −1.2617 | 0.4148 | 0.3692 | 0.5556 | | −1.3796 | −0.3390 | −0.2986 | | | 2.4533 |
| 1951 | 0.5 | 8 | 65.7 | 1.0021 | −1.4515 | 0.4961 | | 0.3697 | | −1.4422 | −0.2495 | −0.4875 | | 0.6985 | 2.1496 |
| 1980 | 0.5 | 8 | 65.7 | 0.8521 | | | 0.2655 | 0.4871 | 0.1908 | −1.6127 | −0.3581 | −0.2340 | | 0.3425 | 4.6906 |
| 1867 | 0.4 | 8 | 66 | 1.0542 | −1.1246 | 0.3121 | 0.4151 | 0.6181 | | −1.3895 | −0.4708 | | −0.3253 | | 4.0601 |
| 1880 | 0.4 | 8 | 66 | | | 0.3309 | 0.2408 | 0.3629 | 0.1753 | −1.6793 | | −0.4986 | −0.4047 | 0.6257 | 7.8218 |
| 1921 | 0.4 | 8 | 66 | 0.9940 | −1.5198 | | | 0.8739 | 0.3562 | −1.3088 | −0.3275 | −0.3179 | −0.3393 | | 7.4496 |
| 1893 | 0.4 | 8 | 66 | 0.9683 | | 0.2900 | | 0.3020 | 0.2384 | −1.5873 | −0.2846 | −0.3210 | −0.3609 | | 5.7177 |
| 1896 | 0.4 | 8 | 66 | 0.9767 | | 0.3951 | 0.2149 | | 0.1972 | −1.5253 | −0.3323 | −0.2874 | −0.3536 | | 4.0289 |
| 1941 | 0.4 | 8 | 66 | 0.9617 | −1.5753 | 0.6798 | 0.1854 | | 0.2482 | −1.3168 | | −0.5597 | | 0.6261 | −0.9800 |
| 1819 | 0.4 | 8 | 66 | 0.9531 | −1.2209 | 0.5419 | 0.2319 | | 0.2444 | −1.2404 | | | −0.3072 | 0.4736 | −1.0654 |
| 1869 | 0.5 | 8 | 66 | 1.0590 | −0.9499 | 0.5465 | 0.3043 | | | −1.3518 | −0.3672 | −0.3220 | −0.3192 | | 2.7486 |
| 1903 | 0.4 | 8 | 66 | 0.9002 | −1.1506 | | | | 0.2653 | −1.2931 | −0.3965 | −0.3613 | −0.3792 | 0.6232 | 5.7766 |
| 1970 | 0.4 | 8 | 66 | 0.8901 | | 0.3655 | 0.3176 | 0.2162 | | −1.6648 | −0.3302 | −0.3146 | | 0.4884 | 2.4690 |
| 1838 | 0.4 | 8 | 66 | 0.8221 | −1.2761 | 0.4474 | 0.3617 | 0.5826 | | −1.4123 | | | −0.3070 | 0.5902 | 0.9129 |
| 1972 | 0.5 | 8 | 66 | 0.9568 | −1.7301 | | | 0.8445 | 0.3270 | −1.3395 | −0.2878 | −0.3777 | | 0.4918 | 5.1042 |
| 1895 | 0.5 | 8 | 66 | 0.8352 | | 0.3956 | 0.1658 | 0.3384 | 0.2319 | −1.6043 | | −0.3953 | −0.3268 | | 4.0488 |
| 1953 | 0.5 | 8 | 66 | 0.8335 | −1.5598 | 0.5942 | 0.3111 | 0.4939 | | −1.4685 | | −0.5354 | | 0.7292 | 0.6623 |
| 1887 | 0.5 | 8 | 66 | 0.8729 | | 0.3999 | | 0.1212 | | −1.7084 | −0.3113 | −0.4113 | −0.4017 | 0.6166 | 5.2818 |
| 1893 | 0.5 | 8 | 66 | 0.9683 | | 0.2900 | | 0.3020 | 0.2384 | −1.5873 | −0.2846 | −0.3210 | −0.3609 | | 5.7177 |
| 1971 | 0.5 | 8 | 66 | 0.9764 | | 0.2843 | 0.2505 | 0.3423 | 0.2006 | −1.5695 | −0.2973 | −0.2594 | | | 3.0619 |
| 1874 | 0.5 | 8 | 66 | 0.8047 | | 0.3042 | 0.2354 | 0.3838 | 0.2081 | −1.5731 | | | −0.3295 | 0.3020 | 2.3340 |
| 1873 | 0.4 | 8 | 66.2 | 0.7948 | | 0.4322 | | 0.2157 | 0.2146 | −1.6909 | | −0.4946 | −0.3709 | 0.4782 | 4.2570 |
| 1888 | 0.4 | 8 | 66.2 | 0.8918 | | 0.2607 | 0.3765 | 0.3163 | | −1.6641 | −0.4844 | | −0.3929 | 0.5164 | 4.4807 |
| 1818 | 0.4 | 8 | 66.2 | 0.9283 | −1.5415 | 0.6868 | | | 0.2726 | −1.3777 | | −0.6232 | −0.3574 | 0.7196 | 1.6563 |
| 1973 | 0.4 | 8 | 66.2 | 0.9996 | −1.6686 | | 0.3682 | 0.9262 | 0.2794 | −1.3276 | −0.4370 | | | 0.4245 | 3.5176 |
| 1838 | 0.5 | 8 | 66.2 | 0.8221 | −1.2761 | 0.4474 | 0.3617 | 0.5826 | | −1.4123 | | | −0.3070 | 0.5902 | 0.9129 |
| 1889 | 0.4 | 8 | 66.2 | 0.7105 | | 0.5155 | 0.2359 | 0.1805 | | −1.7367 | | −0.4858 | −0.3647 | 0.6158 | 3.4212 |
| 1945 | 0.5 | 8 | 66.2 | | −1.3503 | 0.4866 | 0.3090 | | 0.1924 | −1.2905 | −0.1705 | −0.5337 | | 0.7874 | 4.3987 |
| 1889 | 0.5 | 8 | 66.2 | 0.7105 | | 0.5155 | 0.2359 | 0.1805 | | −1.7367 | | −0.4858 | −0.3647 | 0.6158 | 3.4212 |
| 1890 | 0.5 | 8 | 66.2 | 0.8448 | | 0.4479 | 0.2973 | | | −1.6914 | −0.3749 | −0.3552 | −0.4031 | 0.6435 | 4.1756 |
| 1852 | 0.4 | 8 | 66.5 | 0.9921 | −1.6339 | 0.4507 | | 0.6842 | 0.3560 | −1.3313 | | −0.4919 | −0.3066 | | 4.0658 |
| 1894 | 0.4 | 8 | 66.5 | 0.9732 | | 0.1937 | 0.2961 | 0.4324 | 0.2046 | −1.5651 | −0.4263 | | −0.3588 | | 4.9920 |
| 1953 | 0.4 | 8 | 66.5 | 0.8335 | −1.5598 | 0.5942 | 0.3111 | 0.4939 | | −1.4685 | | −0.5354 | | 0.7292 | 0.6623 |
| 1918 | 0.4 | 8 | 66.5 | 0.8072 | −0.9152 | | 0.3033 | | | −1.3900 | −0.4976 | −0.2874 | −0.3764 | 0.7537 | 5.6304 |
| 1904 | 0.4 | 8 | 66.5 | 0.9568 | −1.0458 | | 0.2793 | | 0.2234 | −1.2655 | −0.5303 | | −0.3691 | 0.5469 | 4.1389 |
| 1841 | 0.5 | 8 | 66.5 | 0.8079 | −1.3008 | 0.7392 | 0.2488 | | | −1.4714 | | −0.5937 | −0.3490 | 0.8498 | 1.1350 |
| 1840 | 0.4 | 8 | 66.8 | 1.0229 | −1.0949 | 0.4662 | 0.3881 | | | −1.3915 | −0.5025 | | −0.3754 | 0.6862 | 1.6568 |
| 1934 | 0.4 | 8 | 66.8 | 0.7444 | | | 0.3310 | 0.4711 | | −1.7031 | −0.4571 | −0.2458 | −0.3993 | 0.5893 | 7.3567 |
| 1917 | 0.4 | 8 | 66.8 | 0.5790 | −1.2898 | | 0.2885 | 0.8432 | | −1.4974 | | −0.4462 | −0.3267 | 0.7343 | 6.2068 |
| 1896 | 0.5 | 8 | 66.8 | 0.9767 | | 0.3951 | 0.2149 | | 0.1972 | −1.5253 | −0.3323 | −0.2874 | −0.3536 | | 4.0289 |
| 1905 | 0.5 | 8 | 66.8 | 0.6989 | −1.0852 | | 0.1066 | | 0.2740 | −1.2580 | | −0.4533 | −0.3243 | 0.5996 | 4.1629 |
| 1873 | 0.5 | 8 | 66.8 | 0.7948 | | 0.4322 | | 0.2157 | 0.2146 | −1.6909 | | −0.4946 | −0.3709 | 0.4782 | 4.2570 |
| 1931 | 0.5 | 8 | 66.8 | 0.8053 | | | 0.2210 | | 0.1780 | −1.5344 | −0.4340 | −0.2570 | −0.3942 | 0.5169 | 6.0640 |
| 1894 | 0.5 | 8 | 66.8 | 0.9732 | | 0.1937 | 0.2961 | 0.4324 | 0.2046 | −1.5651 | −0.4263 | | −0.3588 | | 4.9920 |
| 1855 | 0.4 | 8 | 67 | 1.1584 | −1.1076 | 0.4229 | 0.2807 | | 0.2470 | −1.2355 | −0.4320 | | −0.3265 | | 1.7434 |
| 1876 | 0.4 | 8 | 67 | 0.9552 | | 0.3133 | 0.2955 | | 0.1564 | −1.5533 | −0.4555 | | −0.3895 | 0.4401 | 3.2655 |
| 1877 | 0.5 | 8 | 67 | 0.7756 | | 0.5218 | 0.1595 | | 0.1823 | −1.6536 | | −0.4872 | −0.3671 | 0.5232 | 2.8962 |
| 1930 | 0.5 | 8 | 67 | 0.6344 | | | 0.1648 | 0.5602 | 0.2310 | −1.6554 | | −0.3867 | −0.3528 | 0.4223 | 6.3788 |
| 1875 | 0.5 | 8 | 67 | 0.9078 | | 0.4016 | | | 0.1909 | −1.6423 | −0.2895 | −0.4058 | −0.4015 | 0.5181 | 4.6998 |
| 1978 | 0.4 | 8 | 67.3 | 0.8433 | −1.4012 | | 0.3950 | 0.7848 | | −1.4547 | −0.4239 | −0.2852 | | 0.6537 | 4.8068 |
| 1928 | 0.4 | 8 | 67.3 | 0.8235 | | | | 0.4374 | 0.2250 | −1.6483 | −0.3507 | −0.3116 | −0.3994 | 0.4469 | 7.6075 |
| 1875 | 0.4 | 8 | 67.3 | 0.9078 | | 0.4016 | | | 0.1909 | −1.6423 | −0.2895 | −0.4058 | −0.4015 | 0.5181 | 4.6998 |
| 1916 | 0.5 | 8 | 67.3 | 0.8679 | −1.2722 | | 0.4383 | 0.7899 | | −1.4696 | −0.5480 | | −0.3763 | 0.6661 | 6.0480 |
| 1929 | 0.4 | 8 | 67.3 | 0.8569 | | | 0.3120 | 0.5151 | 0.1866 | −1.6256 | −0.4782 | | −0.3917 | 0.3956 | 6.2537 |
| 1928 | 0.5 | 8 | 67.3 | 0.8235 | | | | 0.4374 | 0.2250 | −1.6483 | −0.3507 | −0.3116 | −0.3994 | 0.4469 | 7.6075 |
| 1888 | 0.5 | 8 | 67.3 | 0.8918 | | 0.2607 | 0.3765 | 0.3163 | | −1.6641 | −0.4844 | | −0.3929 | 0.5164 | 4.4807 |
| 1876 | 0.5 | 8 | 67.3 | 0.9552 | | 0.3133 | 0.2955 | | 0.1564 | −1.5533 | −0.4555 | | −0.3895 | 0.4401 | 3.2655 |
| 1922 | 0.4 | 8 | 67.5 | 1.0172 | −1.4850 | | 0.3331 | 0.9492 | 0.3091 | −1.3084 | −0.4577 | | −0.3342 | | 6.0399 |
| 1868 | 0.5 | 8 | 67.5 | 0.8851 | −1.1816 | 0.5443 | 0.2825 | 0.5248 | | −1.4111 | | −0.4391 | −0.2832 | | 2.9679 |
| 1930 | 0.4 | 8 | 67.5 | 0.6344 | | | 0.1648 | 0.5602 | 0.2310 | −1.6554 | | −0.3867 | −0.3528 | 0.4223 | 6.3788 |
| 1929 | 0.5 | 8 | 67.5 | 0.8569 | | | 0.3120 | 0.5151 | 0.1866 | −1.6256 | −0.4782 | | −0.3917 | 0.3956 | 6.2537 |
| 1934 | 0.5 | 8 | 67.5 | 0.7444 | | | 0.3310 | 0.4711 | | −1.7031 | −0.4571 | −0.2458 | −0.3993 | 0.5893 | 7.3567 |
| 1874 | 0.4 | 8 | 67.8 | 0.8047 | | 0.3042 | 0.2354 | 0.3838 | 0.2081 | −1.5731 | | | −0.3295 | 0.3020 | 2.3340 |
| 1935 | 0.4 | 8 | 67.8 | 0.8690 | | | 0.2489 | 0.5531 | 0.2251 | −1.5820 | −0.3934 | −0.2006 | −0.3607 | | 7.1927 |
| 1877 | 0.4 | 8 | 68.5 | 0.7756 | | 0.5218 | 0.1595 | | 0.1823 | −1.6536 | | −0.4872 | −0.3671 | 0.5232 | 2.8962 |
| 1905 | 0.4 | 8 | 68.5 | 0.6989 | −1.0852 | | 0.1066 | | 0.2740 | −12580 | | −0.4533 | −0.3243 | 0.5996 | 4.1629 |
| 1931 | 0.4 | 8 | 69 | 0.8053 | | | 0.2210 | | 0.1780 | −1.5344 | −0.4340 | −0.2570 | −0.3942 | 0.5169 | 6.0640 |
| 1935 | 0.5 | 8 | 69.3 | 0.8690 | | | 0.2489 | 0.5531 | 0.2251 | −1.5820 | −0.3934 | −02006 | −0.3607 | | 7.1927 |
| 1620 | 0.4 | 7 | 65 | 0.8600 | −1.2711 | 0.7082 | | | | −1.4692 | | −0.6260 | −0.3537 | 0.8332 | 1.9179 |
| 1623 | 0.5 | 7 | 65 | 1.0354 | −1.5732 | | | 0.8523 | 0.3520 | −1.2704 | −0.2808 | −0.3145 | | | 5.2078 |
| 1574 | 0.4 | 7 | 65 | 0.9293 | −1.3199 | | 0.4410 | 0.7736 | | −1.4072 | −0.4917 | | | 0.5605 | 3.6219 |
| 1621 | 0.4 | 7 | 65 | 0.9083 | −1.4642 | 0.5888 | | 0.3620 | | −1.4427 | | −0.5783 | | 0.7101 | 1.1181 |
| 1809 | 0.4 | 7 | 65 | 1.0105 | | 0.2758 | | | 0.1987 | −1.5165 | −0.4041 | | −0.3854 | 0.3801 | 3.7338 |
| 1569 | 0.4 | 7 | 65 | 0.8516 | −1.4625 | | 0.2517 | 1.0174 | 0.3467 | −1.2559 | | | −0.2766 | | 4.1640 |
| 1615 | 0.4 | 7 | 65 | 1.0077 | −1.5737 | 0.6543 | | | 0.2714 | −1.3041 | | −0.5821 | | 0.6035 | −0.4603 |
| 1745 | 0.5 | 7 | 65 | | | | 0.3642 | 0.5082 | | −1.6818 | −0.3668 | −0.3287 | −0.4298 | 0.7207 | 10.4670 |
| 1630 | 0.5 | 7 | 65 | 0.8898 | −1.2705 | | | 0.6245 | | −1.4175 | −0.3439 | −0.3620 | | 0.6257 | 5.2220 |
| 1573 | 0.4 | 7 | 65 | 0.8952 | −0.8418 | | 0.3512 | | | −1.3428 | −0.5679 | | −0.3697 | 0.6580 | 4.4318 |
| 1624 | 0.5 | 7 | 65 | 0.9676 | −1.1981 | | | | 0.2660 | −1.2256 | −0.3426 | −0.3447 | | 0.5037 | 3.3040 |
| 1677 | 0.5 | 7 | 65 | 0.9076 | −1.1385 | 0.4275 | | 0.4324 | | −1.3802 | | | −0.3121 | 0.5531 | 1.3821 |
| 1682 | 0.5 | 7 | 65 | 0.9464 | −1.1025 | | | 0.6021 | | −1.4182 | −0.4799 | | −0.3755 | 0.6046 | 6.2038 |
| 1696 | 0.5 | 7 | 65 | 1.0186 | | 0.3857 | 0.2222 | | 0.1893 | −1.4900 | −0.2892 | −0.2820 | | | 1.7521 |
| 1756 | 0.5 | 7 | 65 | 0.8494 | | 0.4141 | 0.2118 | | 0.1917 | −1.4827 | | | −0.3264 | 0.3373 | 0.7348 |
| 1489 | 0.5 | 7 | 65 | 0.9355 | −1.2497 | 0.5332 | 0.2946 | 0.5173 | | −1.3687 | | −0.4257 | | | 1.1896 |
| 1563 | 0.6 | 7 | 65 | 0.9084 | −1.0699 | 0.4242 | 0.3416 | 0.5897 | | −1.3412 | | | −0.2681 | | 1.3925 |
| 1760 | 0.5 | 7 | 65 | 0.9565 | | 0.2586 | 0.3727 | 0.2912 | | −1.5945 | −0.4257 | | | 0.3947 | 1.9372 |
| 1764 | 0.5 | 7 | 65 | 0.9225 | | | 0.3089 | 0.4940 | 0.1882 | −1.5628 | −0.4194 | | | 0.2747 | 3.7319 |
| 1613 | 0.4 | 7 | 65.2 | 1.0394 | −1.2720 | 0.6190 | | | 0.3067 | −1.2636 | | −0.5048 | −0.2993 | | 1.7183 |
| 1659 | 0.5 | 7 | 65.2 | | −1.6153 | | | 0.8578 | 0.3014 | −1.3185 | −0.1583 | −0.4838 | | 0.6488 | 8.9182 |
| 1758 | 0.4 | 7 | 65.2 | 0.9726 | | 0.2568 | 0.3595 | 0.3604 | | −1.5702 | −0.4566 | | −0.3481 | | 4.6268 |
| 1528 | 0.5 | 7 | 65.2 | | −1.0463 | 0.3840 | 0.3439 | 0.6508 | | −1.3754 | | −0.4594 | −0.3213 | | 7.9668 |
| 1535 | 0.4 | 7 | 65.2 | | −1.0026 | | 0.1670 | | 0.2420 | −1.2579 | | −0.4999 | −0.3664 | 0.7479 | 7.3594 |
| 1556 | 0.4 | 7 | 65.2 | 1.2026 | −1.1775 | 0.4207 | 0.2900 | | 0.2439 | −1.1893 | −0.3891 | | | | −0.4368 |
| 1631 | 0.4 | 7 | 65.2 | 0.6439 | −1.1981 | | | 0.7138 | | −1.4779 | | −0.4891 | −0.3346 | 0.7174 | 6.6816 |
| 1486 | 0.4 | 7 | 65.2 | 1.0596 | −1.3305 | 0.6240 | 0.1599 | | 0.2827 | −1.2259 | | −0.4661 | | | −0.5681 |
| 1492 | 0.4 | 7 | 65.2 | 0.8192 | −0.9150 | | 0.0881 | | 0.3057 | −1.1636 | | −0.3726 | −0.2802 | | 4.0076 |
| 1498 | 0.5 | 7 | 65.2 | 0.7082 | −1.0289 | | 0.2750 | 0.8291 | | −1.3861 | | −0.3449 | −0.2739 | | 6.0493 |
| 1620 | 0.5 | 7 | 65.2 | 0.8600 | −1.2711 | 0.7082 | | | | −1.4692 | | −0.6260 | −0.3537 | 0.8332 | 1.9179 |
| 1801 | 0.5 | 7 | 65.2 | | | 0.1594 | | 0.4359 | 0.2419 | −1.5078 | −0.1828 | −0.3926 | −0.3887 | | 10.6505 |
| 1494 | 0.5 | 7 | 65.2 | 0.7748 | −1.1425 | | 0.1229 | | 0.2709 | −1.2022 | | −0.4244 | | 0.5048 | 2.1146 |
| 1592 | 0.5 | 7 | 65.2 | | −1.3678 | 0.0671 | 0.4180 | 0.9442 | 0.2777 | −1.2108 | −0.2863 | | | | 7.4333 |
| 1621 | 0.5 | 7 | 65.2 | 0.9083 | −1.4642 | 0.5888 | | 0.3620 | | −1.4427 | | −0.5783 | | 0.7101 | 1.1181 |
| 1709 | 0.5 | 7 | 65.2 | 0.7197 | | | 0.1553 | 0.5969 | 0.2613 | −1.5659 | | −0.3321 | −0.3199 | | 6.2770 |
| 1744 | 0.5 | 7 | 65.2 | | | | 0.2203 | 0.5805 | 0.2068 | −1.6413 | | −0.4313 | −0.3888 | 0.5557 | 9.2901 |
| 1568 | 0.5 | 7 | 65.2 | 1.0217 | −1.1049 | | 0.2822 | | 0.2260 | −1.2007 | −0.4747 | | | 0.4378 | 1.7568 |
| 1533 | 0.5 | 7 | 65.2 | | −1.0729 | | 0.2753 | | 0.2038 | −1.2222 | −0.2814 | −0.3952 | | 0.6974 | 6.4879 |
| 1626 | 0.5 | 7 | 65.2 | 0.7272 | −1.0924 | | | | 0.2876 | −1.2540 | | −0.4710 | −0.3281 | 0.5891 | 4.4372 |
| 1785 | 0.5 | 7 | 65.2 | 0.8737 | | 0.4050 | | 0.2014 | 0.2127 | −1.6153 | | −0.4534 | | 0.3561 | 2.0200 |
| 1788 | 0.5 | 7 | 65.2 | 0.9404 | | 0.3889 | | 0.0990 | | −1.6258 | −0.2597 | −0.3891 | | 0.4854 | 2.6452 |
| 1699 | 0.5 | 7 | 65.2 | 0.8513 | | 0.4906 | 0.1711 | | 0.1797 | −1.5832 | | −0.4445 | | 0.4002 | 0.7121 |
| 1576 | 0.4 | 7 | 65.5 | 1.0987 | −1.5606 | 0.3630 | 0.2606 | | 0.2788 | | −0.4074 | | −0.2959 | | −3.2349 |
| 1572 | 0.4 | 7 | 65.5 | 0.9592 | −1.0492 | | 0.4073 | 0.7839 | | −1.3824 | −0.5099 | | −0.3249 | | 6.2450 |
| 1622 | 0.4 | 7 | 65.5 | 1.0105 | −0.9682 | | | | 0.2953 | −1.1963 | −0.3804 | −0.2791 | −0.3279 | | 5.5226 |
| 1622 | 0.5 | 7 | 65.5 | 1.0105 | −0.9682 | | | | 0.2953 | −1.1963 | −0.3804 | −0.2791 | −0.3279 | | 5.5226 |
| 1697 | 0.4 | 7 | 65.5 | 0.8806 | | 0.4959 | 0.1400 | | 0.2202 | −1.5252 | | −0.4144 | −0.3217 | | 2.7352 |
| 1557 | 0.4 | 7 | 65.5 | 1.0330 | −1.0820 | 0.5200 | 0.2067 | | 0.2734 | −1.1795 | | | −0.2759 | | −0.7055 |
| 1578 | 0.4 | 7 | 65.5 | 1.0804 | −1.7410 | 0.3782 | 0.2895 | | 0.2549 | | −0.3820 | | | 0.3594 | −5.5541 |
| 1705 | 0.4 | 7 | 65.5 | 0.7862 | | 0.4866 | 0.2443 | 0.1678 | | −1.6635 | | −0.4451 | | 0.4948 | 1.2218 |
| 1714 | 0.4 | 7 | 65.5 | 0.8150 | | | 0.3295 | 0.4431 | | −1.6327 | −0.4035 | −0.2278 | | 0.4603 | 4.7159 |
| 1811 | 0.4 | 7 | 65.5 | 0.8669 | | 0.2828 | | 0.3256 | 0.2373 | −1.5378 | | | −0.3313 | 0.2700 | 2.6939 |
| 1600 | 0.5 | 7 | 65.5 | | −1.1833 | 0.3033 | 0.4208 | 0.6934 | | −1.3783 | | | −0.3500 | 0.7060 | 5.2424 |
| 1789 | 0.5 | 7 | 65.5 | 0.7693 | | 0.5141 | | 0.0986 | | −1.7124 | | −0.5243 | −0.3678 | 0.6129 | 3.8019 |
| 1755 | 0.5 | 7 | 65.5 | 0.8576 | | 0.2904 | 0.2224 | 0.4199 | 0.2321 | −1.5196 | | | −0.3084 | | 2.5771 |
| 1777 | 0.5 | 7 | 65.5 | | | 0.1262 | 0.4327 | 0.4503 | | −1.6048 | −0.4190 | | −0.4261 | 0.6427 | 8.6688 |
| 1813 | 0.5 | 7 | 65.5 | 0.9259 | 0.4270 | | | | 0.2271 | −1.5717 | −0.4227 | | −0.3877 | 0.3403 | 6.3245 |
| 1712 | 0.4 | 7 | 65.7 | 0.8529 | | | 0.3236 | 0.5149 | | −1.5808 | −0.4445 | −0.1753 | −0.3480 | | 7.1849 |
| 1679 | 0.4 | 7 | 65.7 | 1.0146 | −1.0336 | | | | 0.2609 | −1.2351 | −0.4759 | | −0.3684 | 0.4893 | 4.3987 |
| 1496 | 0.5 | 7 | 65.7 | 0.9500 | −1.1507 | | 0.3771 | 0.7741 | | −1.3635 | −0.4176 | −0.2026 | | | 4.9653 |
| 1627 | 0.5 | 7 | 65.7 | 0.8250 | −1.7451 | | | 0.9218 | 0.3507 | −1.3304 | | −0.4624 | | 0.4773 | 4.5522 |
| 1631 | 0.5 | 7 | 65.7 | 0.6439 | −1.1981 | | | 0.7138 | | −1.4779 | | −0.4891 | −0.3346 | 0.7174 | 6.6816 |
| 1741 | 0.5 | 7 | 65.7 | | | | 0.2936 | 0.6158 | 0.2173 | −1.5303 | −0.2863 | −0.2835 | −0.3868 | | 10.9705 |
| 1704 | 0.4 | 7 | 65.7 | 0.7312 | | 0.5680 | 0.2205 | | | −1.6954 | | −0.4981 | −0.3639 | 0.6320 | 2.7272 |
| 1754 | 0.4 | 7 | 65.7 | 1.0192 | | 0.3054 | 0.2931 | | 0.1580 | −1.4878 | −0.3964 | | | 0.3155 | 0.8162 |
| 1786 | 0.5 | 7 | 65.7 | 0.9782 | | 0.3622 | | 0.1918 | | −1.5809 | −0.3092 | −0.3260 | −0.3474 | | 5.3371 |
| 1565 | 0.5 | 7 | 65.7 | 0.8888 | −1.3272 | 0.4360 | 0.3703 | 0.5749 | | −1.3613 | | | | 0.5149 | −0.8755 |
| 1700 | 0.5 | 7 | 65.7 | 0.9679 | | 0.4302 | 0.2845 | | | −1.5409 | −0.3679 | −0.2722 | −0.3434 | | 3.9204 |
| 1763 | 0.5 | 7 | 65.7 | 0.8839 | | | 0.2681 | | 0.1738 | −1.4787 | −0.5023 | | −0.3857 | 0.4398 | 4.9633 |
| 1702 | 0.5 | 7 | 65.7 | 0.9121 | | 0.4286 | 0.2976 | | | −1.6159 | −0.3229 | −0.3313 | | 0.5065 | 1.6325 |
| 1499 | 0.5 | 7 | 65.7 | 0.6040 | −0.8149 | | 0.1902 | | | −1.3484 | | −0.4418 | −0.3181 | 0.7181 | 4.3221 |
| 1752 | 0.5 | 7 | 65.7 | 1.0324 | | 0.3136 | 0.2662 | | 0.1902 | −1.4629 | −0.4254 | | −0.3512 | | 3.2403 |
| 1605 | 0.4 | 7 | 66 | | −1.1893 | | 0.4885 | 0.8291 | | −1.4334 | −0.4601 | | −0.4127 | 0.7880 | 9.3853 |
| 1778 | 0.4 | 7 | 66 | | | | 0.3763 | 0.5621 | 0.1649 | −1.5785 | −0.4001 | | −0.4269 | 0.5328 | 9.5268 |
| 1742 | 0.4 | 7 | 66 | | | | 0.2617 | | 0.1576 | −1.5038 | −0.3417 | −0.3468 | −0.4280 | 0.6752 | 9.3315 |
| 1735 | 0.4 | 7 | 66 | | | 0.4376 | 0.2198 | | 0.1585 | −1.6013 | | −0.5277 | −0.4058 | 0.6777 | 6.6044 |
| 1766 | 0.4 | 7 | 66 | 0.8192 | | | 0.3737 | 0.4759 | | −1.6497 | −0.5191 | | −0.3914 | 0.5134 | 6.3050 |
| 1813 | 0.4 | 7 | 66 | 0.9259 | | | | 0.4270 | 0.2271 | −1.5717 | −0.4227 | | −0.3877 | 0.3403 | 6.3245 |
| 1790 | 0.4 | 7 | 66 | 0.9042 | | | | 0.4807 | 0.2554 | −1.5577 | −0.3371 | −0.2570 | −0.3618 | | 7.4640 |
| 1793 | 0.4 | 7 | 66 | 0.6777 | | | | 0.5082 | 0.2500 | −1.6380 | | −0.4127 | −0.3566 | 0.4104 | 6.6567 |
| 1490 | 0.4 | 7 | 66 | 0.8808 | −1.3437 | 0.7081 | 0.2620 | | | −1.3991 | | −0.5552 | | 0.7398 | −0.9492 |
| 1498 | 0.4 | 7 | 66 | 0.7082 | −1.0289 | | 0.2750 | 0.8291 | | −1.3861 | | −0.3449 | −0.2739 | | 6.0493 |
| 1629 | 0.5 | 7 | 66 | 0.8429 | −0.8818 | | | | | −1.3816 | −0.4281 | −0.3522 | −0.3778 | 0.7331 | 6.2182 |
| 1773 | 0.5 | 7 | 66 | | | 0.0433 | 0.3576 | 0.5989 | 0.2036 | −1.4788 | −0.3479 | | −0.3876 | | 9.7553 |
| 1780 | 0.5 | 7 | 66 | 1.0031 | | 0.3819 | | | 0.2254 | −1.5196 | −0.2823 | −0.3355 | −0.3551 | | 4.4787 |
| 1702 | 0.4 | 7 | 66 | 0.9121 | | 0.4286 | 0.2976 | | | −1.6159 | −0.3229 | −0.3313 | | 0.5065 | 1.6325 |
| 1756 | 0.4 | 7 | 66 | 0.8494 | | 0.4141 | 0.2118 | | 0.1917 | −1.4827 | | | −0.3264 | 0.3373 | 0.7348 |
| 1781 | 0.5 | 7 | 66 | 1.0144 | | 0.2929 | | 0.2644 | 0.2298 | −1.5423 | −0.2387 | −0.3178 | | | 3.2568 |
| 1497 | 0.5 | 7 | 66 | 0.8747 | −0.9745 | | 0.3085 | | | −1.3230 | −0.4463 | −0.2746 | | 0.6404 | 3.1779 |
| 1787 | 0.5 | 7 | 66 | 0.8842 | | 0.4362 | | | | −1.6817 | −0.3095 | −0.4183 | −0.4006 | 0.6275 | 4.7807 |
| 1707 | 0.5 | 7 | 66 | 0.9145 | | | 0.2539 | 0.5194 | 0.2171 | −1.5459 | −0.3494 | −0.1961 | | | 4.7824 |
| 1708 | 0.5 | 7 | 66 | 0.8755 | | | 0.2226 | | 0.1773 | −1.4709 | −0.3797 | −0.2383 | | 0.3847 | 3.5058 |
| 1794 | 0.5 | 7 | 66 | 0.7904 | | | | 0.3633 | | −1.6634 | −0.3882 | −0.3162 | −0.3971 | 0.5784 | 7.7079 |
| 1678 | 0.4 | 7 | 66.2 | 1.0779 | −1.4183 | | | 0.8274 | 0.3432 | −1.2723 | −0.4043 | | −0.3378 | | 6.1309 |
| 1780 | 0.4 | 7 | 66.2 | 1.0031 | | 0.3819 | | | 0.2254 | −1.5196 | −0.2823 | −0.3355 | −0.3551 | | 4.4787 |
| 1538 | 0.5 | 7 | 66.2 | | −0.8343 | | 0.3327 | | | −1.3703 | −0.3956 | −0.3781 | −0.4114 | 0.8959 | 8.9258 |
| 1782 | 0.4 | 7 | 66.2 | 0.9757 | | 0.3848 | | | 0.1912 | −1.5649 | −0.2377 | −0.3803 | | 0.3819 | 2.1371 |
| 1699 | 0.4 | 7 | 66.2 | 0.8513 | | 0.4906 | 0.1711 | | 0.1797 | −1.5832 | | −0.4445 | | 0.4002 | 0.7121 |
| 1563 | 0.5 | 7 | 66.2 | 0.9084 | −1.0699 | 0.4242 | 0.3416 | 0.5897 | | −1.3412 | | | −0.2681 | | 1.3925 |
| 1761 | 0.4 | 7 | 66.2 | 0.7690 | | 0.3807 | 0.2983 | 0.2940 | | −1.5983 | | | −0.3258 | 0.4323 | 1.7193 |
| 1783 | 0.5 | 7 | 66.2 | 0.8757 | | 0.3886 | | 0.2891 | 0.2502 | −1.5899 | | −0.4222 | −0.3309 | | 4.3688 |
| 1784 | 0.5 | 7 | 66.2 | 0.8141 | | 0.4996 | | | 0.2033 | −1.6456 | | −0.5082 | −0.3698 | 0.5048 | 3.3780 |
| 1792 | 0.5 | 7 | 66.2 | 0.8916 | | | | 0.4137 | 0.2246 | −1.5778 | −0.2974 | −0.2906 | | 0.3180 | 4.9704 |
| 1759 | 0.5 | 7 | 66.2 | 0.9304 | | 0.3461 | 0.3516 | | | −1.5887 | −0.4826 | | −0.3898 | 0.5365 | 3.1936 |
| 1567 | 0.4 | 7 | 66.5 | 1.0591 | −1.5482 | | 0.3424 | 0.9341 | 0.3065 | −1.2713 | −0.4130 | | | | 3.8322 |
| 1572 | 0.5 | 7 | 66.5 | 0.9592 | −1.0492 | | 0.4073 | 0.7839 | | −1.3824 | −0.5099 | | −0.3249 | | 6.2450 |
| 1759 | 0.4 | 7 | 66.5 | 0.9304 | | 0.3461 | 0.3516 | | | −1.5887 | −0.4826 | | −0.3898 | 0.5365 | 3.1936 |
| 1491 | 0.5 | 7 | 66.5 | 1.0304 | −1.0325 | | 0.2054 | | 0.2655 | −1.1659 | −0.3879 | −0.2315 | | | 3.0321 |
| 1575 | 0.5 | 7 | 66.5 | 0.6739 | −1.1506 | | 0.3450 | 0.8364 | | −1.4048 | | | −0.2985 | 0.5593 | 3.8749 |
| 1619 | 0.5 | 7 | 66.5 | 0.9473 | −1.0981 | 0.5398 | | 0.4030 | | −1.3996 | | −0.4839 | −0.2901 | | 3.4729 |
| 1569 | 0.5 | 7 | 66.5 | 0.8516 | −1.4625 | | 0.2517 | 1.0174 | 0.3467 | −1.2559 | | | −0.2766 | | 4.1640 |
| 1709 | 0.4 | 7 | 66.5 | 0.7197 | | | 0.1553 | 0.5969 | 0.2613 | −1.5659 | | −0.3321 | −0.3199 | | 6.2770 |
| 1790 | 0.5 | 7 | 66.5 | 0.9042 | | | | 0.4807 | 0.2554 | −1.5577 | −0.3371 | −0.2570 | −0.3618 | | 7.4640 |
| 1765 | 0.4 | 7 | 66.5 | 0.7162 | | | 0.2187 | 0.5847 | 0.2340 | −1.5519 | | | −0.3237 | 0.2780 | 4.3224 |
| 1713 | 0.5 | 7 | 66.5 | 0.7687 | | | 0.2851 | | | −1.5645 | −0.4736 | −0.2484 | −0.3919 | 0.6201 | 6.1633 |
| 1715 | 0.5 | 7 | 66.5 | 0.5647 | | | 0.2332 | 0.5150 | | −1.6799 | | −0.3923 | −0.3457 | 0.5598 | 6.2931 |
| 1704 | 0.5 | 7 | 66.5 | 0.7312 | | 0.5680 | 0.2205 | | | −1.6954 | | −0.4981 | −0.3639 | 0.6320 | 2.7272 |
| 1710 | 0.5 | 7 | 66.5 | 0.6523 | | | 0.1103 | | 0.2183 | −1.4849 | | −0.3980 | −0.3429 | 0.4692 | 4.8824 |
| 1494 | 0.4 | 7 | 66.8 | 0.7748 | −1.1425 | | 0.1229 | | 0.2709 | −1.2022 | | −0.4244 | | 0.5048 | 2.1146 |
| 1784 | 0.4 | 7 | 67 | 0.8141 | | 0.4996 | | | 0.2033 | −1.6456 | | −0.5082 | −0.3698 | 0.5048 | 3.3780 |
| 1715 | 0.4 | 7 | 67 | 0.5647 | | | 0.2332 | 0.5150 | | −1.6799 | | −0.3923 | −0.3457 | 0.5598 | 6.2931 |
| 1499 | 0.4 | 7 | 67 | 0.6040 | −0.8149 | | 0.1902 | | | −1.3484 | | −0.4418 | −0.3181 | 0.7181 | 4.3221 |
| 1710 | 0.4 | 7 | 67 | 0.6523 | | | 0.1103 | | 0.2183 | −1.4849 | | −0.3980 | −0.3429 | 0.4692 | 4.8824 |
| 1763 | 0.4 | 7 | 67 | 0.8839 | | | 0.2681 | | 0.1738 | −1.4787 | −0.5023 | | −0.3857 | 0.4398 | 4.9633 |
| 1791 | 0.5 | 7 | 67 | 0.8389 | | | | | 0.2083 | −1.5229 | −0.3788 | −0.3056 | −0.3937 | 0.4881 | 6.4428 |
| 1762 | 0.4 | 7 | 67.3 | 0.9203 | | | 0.2894 | 0.5523 | 0.2175 | −1.5521 | −0.4493 | | −0.3590 | | 6.3303 |
| 1706 | 0.4 | 7 | 67.3 | 0.9089 | | | 0.1979 | | 0.2142 | −1.4158 | −0.4157 | −0.1992 | −0.3485 | | 5.7840 |
| 1495 | 0.5 | 7 | 67.3 | 0.9401 | −0.6610 | | 0.2841 | | | −1.2797 | −0.4791 | −0.1927 | −0.3166 | | 5.4323 |
| 1712 | 0.5 | 7 | 67.3 | 0.8529 | | | 0.3236 | 0.5149 | | −1.5808 | −0.4445 | −0.1753 | −0.3480 | | 7.1849 |
| 1762 | 0.5 | 7 | 67.3 | 0.9203 | | | 0.2894 | 0.5523 | 0.2175 | −1.5521 | −0.4493 | | −0.3590 | | 6.3303 |
| 1570 | 0.4 | 7 | 67.3 | 0.7905 | −0.9233 | | 0.1703 | | 0.2662 | −1.1657 | | | −0.2947 | 0.4228 | 1.7978 |
| 1566 | 0.4 | 7 | 67.5 | 1.0439 | −0.9065 | | 0.2429 | | 0.2574 | −1.1848 | −0.4917 | | −0.3248 | | 4.2112 |
| 1713 | 0.4 | 7 | 67.5 | 0.7687 | | | 0.2851 | | | −1.5645 | −0.4736 | −0.2484 | −0.3919 | 0.6201 | 6.1633 |
| 1706 | 0.5 | 7 | 67.5 | 0.9089 | | | 0.1979 | | 0.2142 | −1.4158 | −0.4157 | −0.1992 | −0.3485 | | 5.7840 |
| 1755 | 0.4 | 7 | 67.5 | 0.8576 | | 0.2904 | 0.2224 | 0.4199 | 0.2321 | −1.5196 | | | −0.3084 | | 2.5771 |
| 1697 | 0.5 | 7 | 67.5 | 0.8806 | | 0.4959 | 0.1400 | | 0.2202 | −1.5252 | | −0.4144 | −0.3217 | | 2.7352 |
| 1500 | 0.4 | 7 | 67.8 | 0.6553 | −1.3444 | | 0.3014 | 0.8269 | | −1.4395 | | −0.4183 | | 0.6408 | 4.1221 |
| 1752 | 0.4 | 7 | 67.8 | 1.0324 | | 0.3136 | 0.2662 | | 0.1902 | −1.4629 | −0.4254 | | −0.3512 | | 3.2403 |
| 1793 | 0.5 | 7 | 67.8 | 0.6777 | | | | 0.5082 | 0.2500 | −1.6380 | | −0.4127 | −0.3566 | 0.4104 | 6.6567 |
| 1766 | 0.5 | 7 | 67.8 | 0.8192 | | | 0.3737 | 0.4759 | | −1.6497 | −0.5191 | | −0.3914 | 0.5134 | 6.3050 |
| 1626 | 0.4 | 7 | 68 | 0.7272 | −1.0924 | | | | 0.2876 | −1.2540 | | −0.4710 | −0.3281 | 0.5891 | 4.4372 |
| 1791 | 0.4 | 7 | 68.3 | 0.8389 | | | | | 0.2083 | −1.5229 | −0.3788 | −0.3056 | −0.3937 | 0.4881 | 6.4428 |
| 1338 | 0.4 | 6 | 65 | 1.0737 | | 0.2787 | | | 0.2247 | −1.4414 | −0.3825 | | −0.3529 | | 3.6819 |
| 1456 | 0.4 | 6 | 65 | 0.9779 | | | | 0.4639 | 0.2514 | −1.5122 | −0.4015 | | −0.3601 | | 6.3893 |
| 1164 | 0.4 | 6 | 65 | | −1.1089 | | 0.3993 | 0.8622 | | −1.3763 | | | −0.3388 | 0.6677 | 6.8210 |
| 1420 | 0.5 | 6 | 65 | | −0.9108 | | 0.4629 | 0.8348 | | −1.3321 | −0.4039 | | −0.3579 | | 10.0978 |
| 1465 | 0.5 | 6 | 65 | 1.0067 | −1.3551 | | 0.2268 | | 0.2870 | | −0.4610 | | −0.2976 | | −0.9391 |
| 1314 | 0.4 | 6 | 65 | 0.8543 | | 0.5471 | 0.2107 | | | −1.5424 | | −0.4111 | −0.3068 | | 2.4762 |
| 1414 | 0.5 | 6 | 65 | | −0.5064 | | 0.3153 | | | −1.2339 | −0.3568 | −0.2869 | −0.3495 | | 9.4284 |
| 1416 | 0.5 | 6 | 65 | | −0.7618 | | 0.1544 | | 0.2770 | −1.1371 | | −0.4120 | −0.3192 | | 7.9159 |
| 1034 | 0.5 | 6 | 65 | 0.9443 | −0.9496 | 0.5296 | | | | −1.3161 | | | −0.3097 | 0.5605 | −0.2281 |
| 1426 | 0.5 | 6 | 65 | 0.8405 | −0.9224 | | | | 0.3163 | −1.1624 | | −0.3886 | −0.2840 | | 4.2424 |
| 1152 | 0.4 | 6 | 65 | 0.8600 | −0.9839 | | 0.1803 | | 0.2653 | −1.1193 | | | | 0.3512 | 0.0249 |
| 1208 | 0.5 | 6 | 65 | 0.7595 | | | | 0.4779 | 0.2466 | −1.5745 | | −0.3789 | | 0.3003 | 4.3845 |
| 1332 | 0.5 | 6 | 65 | 1.0230 | | 0.3633 | | 0.1560 | | −1.5334 | −0.2643 | −0.3248 | | | 2.9570 |
| 1194 | 0.5 | 6 | 65 | 0.6485 | | | 0.2876 | 0.5385 | | −1.5766 | | | −0.3180 | 0.4200 | 4.2043 |
| 1320 | 0.5 | 6 | 65 | 0.8436 | | 0.3721 | 0.2891 | 0.3324 | | −1.5213 | | | −0.2932 | | 1.9738 |
| 1443 | 0.5 | 6 | 65 | 0.9654 | | | 0.2935 | 0.5203 | 0.2104 | −1.5152 | −0.4038 | | | | 3.9376 |
| 1202 | 0.4 | 6 | 65.2 | | | | 0.3306 | | 0.1498 | −1.4155 | −0.4224 | | −0.4225 | 0.5929 | 8.2059 |
| 1407 | 0.4 | 6 | 65.2 | 1.0039 | −1.1168 | | 0.4136 | 0.7674 | | −1.3395 | −0.4678 | | | | 4.0669 |
| 1045 | 0.4 | 6 | 65.2 | 0.8057 | | 0.5348 | 0.2298 | | | −1.6243 | | −0.4564 | | 0.5096 | 0.5788 |
| 1173 | 0.4 | 6 | 65.2 | 0.8386 | −0.9203 | | | | 0.2874 | −1.1553 | | | −0.2987 | 0.3942 | 2.1188 |
| 1293 | 0.5 | 6 | 65.2 | 1.0023 | −1.1629 | 0.5291 | | 0.3896 | | −1.3510 | | −0.4715 | | | 1.6417 |
| 1294 | 0.5 | 6 | 65.2 | 0.9843 | −0.9231 | 0.6358 | | | | −1.3453 | | −0.4930 | −0.2874 | | 2.0236 |
| 1199 | 0.5 | 6 | 65.2 | | | | 0.1659 | | 0.1929 | −1.4663 | | −0.4451 | −0.3798 | 0.6137 | 7.8233 |
| 1186 | 0.4 | 6 | 65.2 | 0.7301 | | | 0.1239 | | 0.2135 | −1.4324 | | −0.3650 | | 0.3609 | 2.7383 |
| 1049 | 0.5 | 6 | 65.2 | 0.8067 | | 0.4616 | 0.2755 | | | −1.5244 | | | −0.3228 | 0.4520 | 0.4947 |
| 1061 | 0.5 | 6 | 65.2 | 0.8923 | | 0.4673 | | | 0.2017 | −1.5716 | | −0.4663 | | 0.3805 | 1.1997 |
| 1193 | 0.5 | 6 | 65.2 | 0.7395 | | | 0.1645 | | 0.2205 | −1.3708 | | | −0.3138 | 0.3260 | 2.6822 |
| 1190 | 0.5 | 6 | 65.2 | 0.8844 | | | 0.3700 | 0.4497 | | −1.5842 | −0.4614 | | | 0.3936 | 3.7741 |
| 1316 | 0.4 | 6 | 65.5 | 1.0175 | | 0.2574 | 0.3603 | 0.3255 | | −1.5278 | −0.4108 | | | | 2.2651 |
| 1199 | 0.4 | 6 | 65.5 | | | | 0.1659 | | 0.1929 | −1.4663 | | −0.4451 | −0.3798 | 0.6137 | 7.8233 |
| 1207 | 0.4 | 6 | 65.5 | 0.8054 | | | | | | −1.5553 | −0.4087 | −0.3108 | −0.3913 | 0.6041 | 6.7049 |
| 1315 | 0.4 | 6 | 65.5 | 1.0736 | | 0.3066 | 0.2725 | | 0.1830 | −1.4275 | −0.3802 | | | | 0.9768 |
| 1187 | 0.4 | 6 | 65.5 | 0.6447 | | | 0.2415 | 0.4856 | | −1.6181 | | −0.3604 | | 0.4527 | 4.0622 |
| 1411 | 0.5 | 6 | 65.5 | 0.7609 | −0.9661 | | 0.3257 | 0.8290 | | −1.3338 | | | −0.2631 | | 4.1886 |
| 1451 | 0.5 | 6 | 65.5 | | | | 0.3550 | 0.6247 | 0.2065 | −1.4774 | −0.3544 | | −0.3873 | | 10.0113 |
| 1185 | 0.4 | 6 | 65.5 | 0.8380 | | | 0.2858 | | | −1.5014 | −0.4196 | −0.2319 | | 0.4909 | 3.6279 |
| 1214 | 0.4 | 6 | 65.5 | 0.7789 | | | | 0.5170 | 0.2599 | −1.5196 | | | −0.3264 | 0.2493 | 4.5339 |
| 1164 | 0.5 | 6 | 65.5 | | −1.1089 | | 0.3993 | 0.8622 | | −1.3763 | | | −0.3388 | 0.6677 | 6.8210 |
| 1445 | 0.5 | 6 | 65.5 | 0.8994 | | | 0.3563 | 0.5157 | | −1.5547 | −0.4909 | | −0.3475 | | 6.4228 |
| 1209 | 0.5 | 6 | 65.5 | 0.6814 | | | | | 0.2319 | −1.4832 | | −0.4155 | −0.3461 | 0.4576 | 5.1681 |
| 1331 | 0.5 | 6 | 65.5 | 1.0455 | | 0.3728 | | | 0.2180 | −1.4810 | −0.2375 | −0.3308 | | | 2.1921 |
| 1444 | 0.5 | 6 | 65.5 | 0.9613 | | | 0.2383 | | 0.2069 | −1.3870 | −0.4722 | | −0.3476 | | 4.9343 |
| 1446 | 0.5 | 6 | 65.5 | 0.7677 | | | 0.2074 | 0.6092 | 0.2550 | −1.5015 | | | −0.3049 | | 4.4620 |
| 1168 | 0.5 | 6 | 65.5 | 0.6482 | −0.7994 | | | | | −1.3500 | | −0.4726 | −0.3242 | 0.7080 | 4.8418 |
| 1163 | 0.5 | 6 | 65.7 | | −0.8149 | | 0.2405 | | 0.2316 | −1.1424 | | | −0.3433 | 0.5703 | 5.1715 |
| 1167 | 0.4 | 6 | 65.7 | 0.7225 | −1.2447 | | | 0.6908 | | −1.4138 | | −0.4617 | | 0.6207 | 4.5580 |
| 1422 | 0.5 | 6 | 65.7 | 1.0548 | −1.0407 | | | | 0.2921 | −1.1543 | −0.3371 | −0.2799 | | | 3.3758 |
| 1454 | 0.5 | 6 | 65.7 | 0.8964 | | | | 0.4071 | | −1.5458 | −0.3788 | −0.2457 | −0.3480 | | 7.5503 |
| 1210 | 0.4 | 6 | 65.7 | 0.6198 | | | | 0.4317 | | −1.6549 | | −0.4305 | −0.3502 | 0.5567 | 6.6786 |
| 1153 | 0.5 | 6 | 65.7 | 0.7426 | −1.2063 | | 0.3534 | 0.8230 | | −1.3573 | | | | 0.4882 | 2.0672 |
| 1455 | 0.5 | 6 | 65.7 | 0.7581 | | | | 0.5462 | 0.2781 | −1.5523 | | −0.3581 | −0.3244 | | 6.5450 |
| 1402 | 0.5 | 6 | 65.7 | 0.9857 | −0.7410 | | 0.2913 | | | −1.2374 | −0.4399 | −0.1947 | | | 3.3399 |
| 1439 | 0.5 | 6 | 65.7 | 0.8911 | | | 0.2720 | | | −1.4252 | −0.4619 | −0.1758 | −0.3362 | | 5.8585 |
| 1314 | 0.5 | 6 | 65.7 | 0.8543 | | 0.5471 | 0.2107 | | | −1.5424 | | −0.4111 | −0.3068 | | 2.4762 |
| 1060 | 0.5 | 6 | 65.7 | 0.9500 | | 0.4182 | | | | −1.6034 | −0.2583 | −0.3949 | | 0.4943 | 2.2377 |
| 1205 | 0.5 | 6 | 65.7 | 0.9070 | | | | | 0.2077 | −1.4575 | −0.3247 | −0.2866 | | 0.3579 | 3.8907 |
| 1207 | 0.5 | 6 | 65.7 | 0.8054 | | | | | | −1.5553 | −0.4087 | −0.3108 | −0.3913 | 0.6041 | 6.7049 |
| 1439 | 0.4 | 6 | 66 | 0.8911 | | | 0.2720 | | | −1.4252 | −0.4619 | −0.1758 | −0.3362 | | 5.8585 |
| 1150 | 0.4 | 6 | 66 | 0.6794 | −0.8779 | | 0.2033 | | | −1.2905 | | −0.4163 | | 0.6278 | 2.3104 |
| 1158 | 0.4 | 6 | 66 | | −0.7769 | | 0.2338 | | | −1.3369 | | −0.4864 | −0.3547 | 0.8391 | 7.1060 |
| 1290 | 0.5 | 6 | 66 | | −0.9271 | 0.2546 | 0.4058 | 0.7247 | | −1.2931 | | | −0.3081 | | 6.4301 |
| 1453 | 0.5 | 6 | 66 | 0.9335 | | | | | 0.2397 | −1.4121 | −0.3673 | −0.2457 | −0.3509 | | 6.1503 |
| 1408 | 0.5 | 6 | 66 | 0.9920 | −0.6322 | | 0.3195 | | | −1.2581 | −0.5293 | | −0.3175 | | 4.6062 |
| 1403 | 0.4 | 6 | 66 | 0.8704 | −0.9872 | | 0.1047 | | 0.2993 | −1.1282 | | −0.3600 | | | 2.2241 |
| 1438 | 0.5 | 6 | 66 | 0.8985 | | | 0.3245 | 0.4799 | | −1.5421 | −0.4006 | −0.1739 | | | 4.8438 |
| 1185 | 0.5 | 6 | 66 | 0.8380 | | | 0.2858 | | | −1.5014 | −0.4196 | −0.2319 | | 0.4909 | 3.6279 |
| 1191 | 0.5 | 6 | 66 | 0.8465 | | | 0.3282 | | | −1.5098 | −0.5379 | | −0.3846 | 0.5453 | 5.0882 |
| 1429 | 0.4 | 6 | 66.2 | 1.0876 | −0.9052 | | | | 0.2874 | −1.1682 | −0.4473 | | −0.3290 | | 4.4431 |
| 1191 | 0.4 | 6 | 66.2 | 0.8465 | | | 0.3282 | | | −1.5098 | −0.5379 | | −0.3846 | 0.5453 | 5.0882 |
| 1335 | 0.5 | 6 | 66.2 | 0.9103 | | 0.4771 | | | 0.2374 | −1.5227 | | −0.4353 | −0.3258 | | 3.1753 |
| 1189 | 0.5 | 6 | 66.2 | 0.9491 | | | 0.2664 | | 0.1744 | −1.4191 | −0.4434 | | | 0.3170 | 2.5119 |
| 1453 | 0.4 | 6 | 66.5 | 0.9335 | | | | | 0.2397 | −1.4121 | −0.3673 | −0.2457 | −0.3509 | | 6.1503 |
| 1410 | 0.4 | 6 | 66.5 | 0.8685 | −0.8181 | | 0.1485 | | 0.2915 | −1.1094 | | | −0.2676 | | 2.0067 |
| 1049 | 0.4 | 6 | 66.5 | 0.8067 | | 0.4616 | 0.2755 | | | −1.5244 | | | −0.3228 | 0.4520 | 0.4947 |
| 1203 | 0.5 | 6 | 66.5 | | | | 0.4282 | 0.5251 | | −1.6009 | −0.4396 | | −0.4241 | 0.6357 | 9.4282 |
| 1194 | 0.4 | 6 | 66.5 | 0.6485 | | | 0.2876 | 0.5385 | | −1.5766 | | | −0.3180 | 0.4200 | 4.2043 |
| 1168 | 0.4 | 6 | 66.8 | 0.6482 | −0.7994 | | | | | −1.3500 | | −0.4726 | −0.3242 | 0.7080 | 4.8418 |
| 1209 | 0.4 | 6 | 66.8 | 0.6814 | | | | | 0.2319 | −1.4832 | | −0.4155 | −0.3461 | 0.4576 | 5.1681 |
| 1452 | 0.5 | 6 | 66.8 | 0.9483 | | | | 0.4460 | 0.2475 | −1.5177 | −0.2921 | −0.2530 | | | 5.0483 |
| 1446 | 0.4 | 6 | 66.8 | 0.7677 | | | 0.2074 | 0.6092 | 0.2550 | −1.5015 | | | −0.3049 | | 4.4620 |
| 1206 | 0.5 | 6 | 66.8 | 0.8570 | | | | 0.3355 | | −1.5898 | −0.3359 | −0.2987 | | 0.4534 | 5.0792 |
| 1319 | 0.4 | 6 | 67 | 0.9160 | | 0.4098 | 0.1941 | | 0.2171 | −1.4153 | | | −0.3017 | | 0.8410 |
| 1441 | 0.4 | 6 | 67 | 0.7539 | | | 0.0945 | | 0.2508 | −1.3760 | | −0.3390 | −0.3049 | | 4.6574 |
| 1166 | 0.4 | 6 | 67 | 0.8072 | −1.1479 | | | | 0.2862 | −1.1961 | | −0.4437 | | 0.4912 | 2.4023 |
| 1437 | 0.5 | 6 | 67 | 0.9516 | | | 0.2057 | | 0.2060 | −1.3873 | −0.3719 | −0.1963 | | | 3.5203 |
| 1441 | 0.5 | 6 | 67 | 0.7539 | | | 0.0945 | | 0.2508 | −1.3760 | | −0.3390 | −0.3049 | | 4.6574 |
| 1188 | 0.5 | 6 | 67 | 0.5851 | | | 0.1785 | | | −1.5176 | | −0.4024 | −0.3353 | 0.5941 | 4.8960 |
| 1212 | 0.4 | 6 | 67.3 | 0.9416 | | | | | 0.2107 | −1.4513 | −0.4500 | | −0.3833 | 0.3839 | 5.2092 |
| 1193 | 0.4 | 6 | 67.3 | 0.7395 | | | 0.1645 | | 0.2205 | −1.3708 | | | −0.3138 | 0.3260 | 2.6822 |
| 1154 | 0.5 | 6 | 67.3 | 0.7003 | −0.6770 | | 0.2476 | | | −1.2540 | | | −0.2916 | 0.5492 | 1.9924 |
| 1444 | 0.4 | 6 | 67.5 | 0.9613 | | | 0.2383 | | 0.2069 | −1.3870 | −0.4722 | | −0.3476 | | 4.9343 |
| 1405 | 0.5 | 6 | 67.8 | 0.7390 | −0.5713 | | 0.1776 | | | −1.2448 | | −0.3448 | −0.2665 | | 4.1808 |
| 598 | 0.4 | 5 | 65 | 0.8089 | | | 0.1726 | | 0.2169 | −1.3317 | | | | 0.2420 | 0.8455 |
| 720 | 0.4 | 5 | 65 | 0.9020 | | 0.5250 | 0.2208 | | | −1.5084 | | −0.3920 | | | 0.6358 |
| 740 | 0.5 | 5 | 65 | 0.8967 | | 0.5242 | | | | −1.5405 | | −0.4434 | −0.3114 | | 3.1371 |
| 632 | 0.5 | 5 | 65 | 0.8714 | | | | | | −1.4899 | −0.3554 | −0.2948 | | 0.4783 | 4.1787 |
| 634 | 0.5 | 5 | 65 | 0.7619 | | | | | 0.2286 | −1.4285 | | −0.3840 | | 0.3473 | 3.0367 |
| 685 | 0.5 | 5 | 65 | 1.0388 | −0.9924 | 0.6213 | | | | −1.2976 | | −0.4801 | | | 0.2477 |
| 917 | 0.5 | 5 | 65 | | −0.7450 | | 0.3191 | | 0.2254 | −1.0661 | −0.3279 | | | | 6.0212 |
| 657 | 0.5 | 5 | 65.2 | 1.0787 | −1.3551 | 0.3888 | 0.3507 | | | | −0.4056 | | | | −5.0165 |
| 718 | 0.4 | 5 | 65.2 | 0.9637 | | 0.3957 | 0.2049 | | 0.2085 | −1.3879 | | | | | −0.9191 |
| 742 | 0.5 | 5 | 65.2 | 0.9689 | | | | 0.3930 | | −1.5029 | −0.4386 | | −0.3478 | | 6.5139 |
| 705 | 0.5 | 5 | 65.2 | 0.7212 | | | 0.2787 | 0.5691 | | −1.4993 | | | −0.2875 | | 4.4005 |
| 579 | 0.4 | 5 | 65.5 | 0.7259 | −0.8590 | | | | | −1.2891 | | −0.4483 | | 0.6153 | 2.8248 |
| 615 | 0.5 | 5 | 65.5 | 0.6270 | | | | | | −1.5177 | | −0.4318 | −0.3401 | 0.5872 | 5.3759 |
| 590 | 0.4 | 5 | 65.5 | 0.6745 | | | 0.2327 | | | −1.4036 | | | −0.3074 | 0.4554 | 2.6660 |
| 646 | 0.5 | 5 | 65.5 | 0.7916 | −0.5040 | | 0.2284 | | | −1.1896 | | | −0.2561 | | 2.3066 |
| 721 | 0.5 | 5 | 65.5 | 1.0036 | | | 0.2454 | | 0.1992 | −1.3570 | −0.4271 | | | | 2.6741 |
| 590 | 0.5 | 5 | 65.5 | 0.6745 | | | 0.2327 | | | −1.4036 | | | −0.3074 | 0.4554 | 2.6660 |
| 635 | 0.4 | 5 | 65.7 | 0.6996 | | | | 0.3985 | | −1.5880 | | −0.3998 | | 0.4494 | 4.4305 |
| 703 | 0.4 | 5 | 65.7 | 0.9390 | | | 0.3048 | | | −1.3990 | −0.5095 | | −0.3360 | | 5.0937 |
| 745 | 0.4 | 5 | 65.7 | 0.7764 | | | | | 0.2617 | −1.3772 | | −0.3553 | −0.3086 | | 4.9122 |
| 772 | 0.5 | 5 | 65.7 | | −0.6123 | | 0.3022 | | | −1.2207 | | | −0.3335 | 0.6670 | 4.9843 |
| 612 | 0.5 | 5 | 65.7 | 0.9105 | | | | | | −1.4831 | −0.4813 | | −0.3825 | 0.5034 | 5.4469 |
| 741 | 0.5 | 5 | 65.7 | 1.0052 | | | | | 0.2366 | −1.3739 | −0.4288 | | −0.3504 | | 5.1663 |
| 741 | 0.4 | 5 | 66 | 1.0052 | | | | | 0.2366 | −1.3739 | −0.4288 | | −0.3504 | | 5.1663 |
| 899 | 0.5 | 5 | 66 | | −0.4436 | | 0.3714 | | | −1.1904 | −0.4196 | | −0.3528 | | 8.4458 |
| 832 | 0.4 | 5 | 66 | | | | 0.2210 | | | −1.4969 | | −0.4460 | −0.3691 | 0.7146 | 7.5597 |
| 723 | 0.5 | 5 | 66 | 0.9338 | | | 0.2762 | | | −1.3962 | −0.4184 | −0.1751 | | | 3.6687 |
| 671 | 0.5 | 5 | 66.2 | 0.7774 | −0.5578 | | | | | −1.2497 | | −0.3753 | −0.2730 | | 4.6838 |
| 615 | 0.4 | 5 | 66.5 | 0.6270 | | | | | | −1.5177 | | −0.4318 | −0.3401 | 0.5872 | 5.3759 |
| 669 | 0.4 | 5 | 66.5 | 0.9065 | −0.8204 | | | | 0.3086 | −1.1045 | | | −0.2728 | | 2.2797 |
| 654 | 0.4 | 5 | 66.5 | 0.9200 | −0.8888 | | 0.1612 | | 0.2867 | −1.0754 | | | | | 0.3400 |
| 704 | 0.4 | 5 | 66.5 | 0.8046 | | | 0.1474 | | 0.2442 | −1.3056 | | | −0.2906 | | 2.7654 |
| 705 | 0.4 | 5 | 66.5 | 0.7212 | | | 0.2787 | 0.5691 | | −1.4993 | | | −0.2875 | | 4.4005 |
| 597 | 0.5 | 5 | 66.8 | 0.9107 | | | 0.3266 | | | −1.4506 | −0.4798 | | | 0.4242 | 2.6502 |
| 764 | 0.5 | 5 | 67 | 0.9757 | | | | | 0.2323 | −1.3808 | −0.3216 | −0.2439 | | | 3.8820 |
| 901 | 0.5 | 5 | 67.3 | | −0.8795 | | 0.3871 | 0.8641 | | −1.2931 | | | −0.3014 | | 7.7103 |
| 613 | 0.4 | 5 | 67.3 | 0.7863 | | | | | 0.2413 | −1.3610 | | | −0.3169 | 0.2986 | 2.9896 |
| 745 | 0.5 | 5 | 67.3 | 0.7764 | | | | | 0.2617 | −1.3772 | | −0.3553 | −0.3086 | | 4.9122 |
| 706 | 0.5 | 5 | 67.8 | 0.7072 | | | 0.1703 | | | −1.3807 | | −0.3274 | −0.2857 | | 4.6125 |
| 257 | 0.4 | 4 | 65 | 0.8422 | | | | | 0.2613 | −1.3021 | | | −0.2953 | | 3.0395 |
| 339 | 0.5 | 4 | 65 | 0.7575 | | | 0.1815 | | | −1.3564 | | −0.3135 | | | 2.8192 |
| 304 | 0.5 | 4 | 65 | 0.8313 | −0.6339 | | | | | −1.2085 | | −0.3667 | | | 2.9611 |
| 341 | 0.4 | 4 | 65.2 | 0.7592 | | | 0.2190 | | | −1.3129 | | | −0.2730 | | 2.7836 |
| 333 | 0.5 | 4 | 65.2 | 0.9636 | | | | | | −1.3888 | −0.3567 | −0.2376 | | | 4.2105 |
| 133 | 0.5 | 3 | 66 | 0.7970 | | | | | | −1.3564 | | −0.3442 | | | 3.2864 |

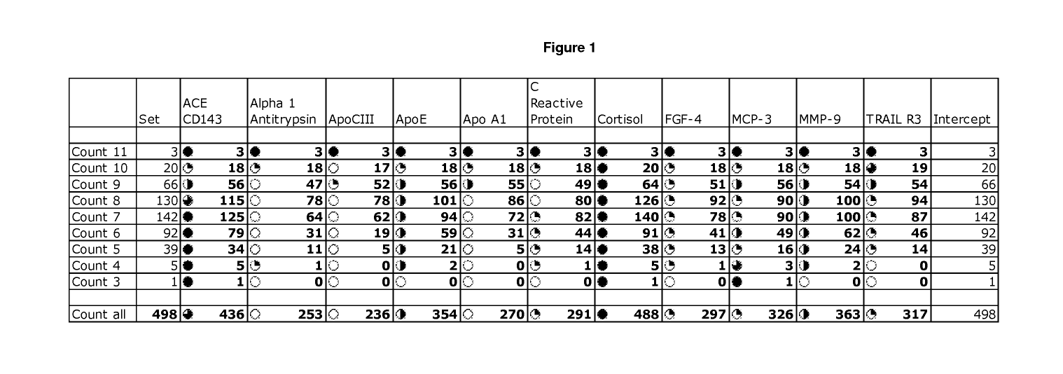

To identify the most informative biomarkers the models were partitioned according to the number of markers in the model. Within each partition, the number of models that included each biomarker was counted. The tabulated results for all 498 models are shown in FIG. 1. FIG. 1 shows the marker frequency for all models above 65% accuracy.

Markers that occur in a high percentage of the AD biomarker sets with at least 65% accuracy (including multiple cut offs) include ACE, cortisol, ApoE, MCP-3, and MMP-9.

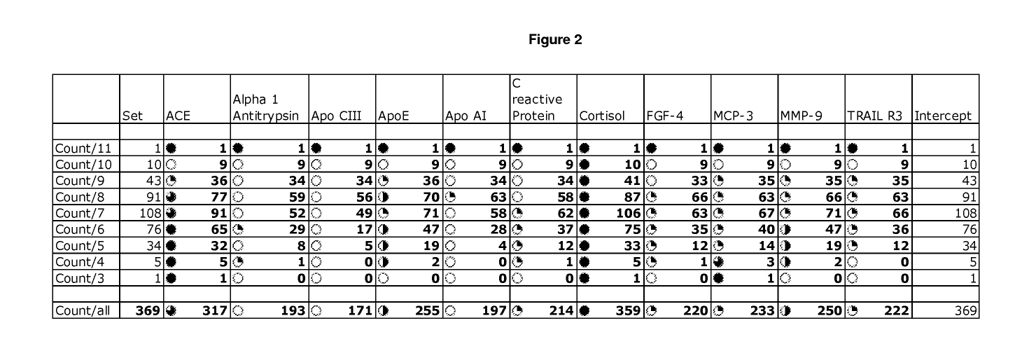

Since some specific algorithms occur more than once due to having more than one cutoff with at least 65% Accuracy, and this might skew the results, only the unique models were also tabulated. The results are shown in FIG. 2. FIG. 2 shows the marker frequency in all unique models above 65% accuracy (multiple cutoffs eliminated).

Amount of circle shading is used to visualize the frequency with which a given marker appeared in models of the various sizes with longer shading indicating the higher frequency. Whether all models are included in the analysis or only the unique models are included, the “core” biomarkers which appear the most frequently are the same and include: cortisol, ACE (CD143), Apo E, MCP-3, and MMP-9.

Accordingly, in various embodiments of the invention for use with any of the methods, kits, or surfaces described herein, the set of AD diagosis biomakers comprises at least one of the following AD diagnosis biomarkers: ACE, cortisol, Apo E, MCP-3, and MMP-9. In some embodiments, the set of AD diagosis biomakers comprises at least two of the following AD diagnosis biomarkers: ACE, cortisol, Apo E, MCP-3, and MMP-9 (e.g. ACE and cortisol, ACE and Apo E, ACE and MCP-3, ACE and MMP-9, cortisol and Apo E, cortisol and MCP-3, cortisol and MMP-9, Apo E and MCP-3, Apo E and MMP-9, or MCP-3 and MMP-9). In some embodiments, the set of AD diagosis biomakers comprises at least three of the following AD diagnosis biomarkers: ACE, cortisol, Apo E, MCP-3, and MMP-9 (e.g. ACE, cortisol, and Apo E; ACE, cortisol, and MCP-3; ACE, cortisol, and MMP-9; ACE, Apo E, and MCP-3; ACE, Apo E, and MMP-9; ACE, MCP-3, and MMP-9; cortisol, Apo E, and MCP-3; cortisol, Apo E, and MMP-9; cortisol, MCP-3, and MMP-9; or Apo E, MCP-3, and MMP-9). In some embodiments, the set of AD diagosis biomakers comprises at least four of the following AD diagnosis biomarkers: ACE, cortisol, Apo E, MCP-3, and MMP-9 (e.g. ACE, cortisol, Apo E, and MCP-3; ACE, cortisol, Apo E, and MMP-9; ACE, cortisol, MCP-3, and MMP-9; ACE, Apo E, MCP-3, and MMP-9; or cortisol, Apo E, MCP-3, and MMP-9). In some embodiments, the set of AD diagosis biomakers comprises at least the following AD diagnosis biomarkers: ACE, cortisol, Apo E, MCP-3, and MMP-9. In some embodiments, the set of AD diagosis biomakers comprises ACE. In some embodiments, the set of AD diagosis biomakers comprises cortisol. In some embodiments, the set of AD diagosis biomakers comprises Apo E. In some embodiments, the set of AD diagosis biomakers comprises MCP-3. In some embodiments, the set of AD diagosis biomakers comprises MMP-9.

### REFERENCES

R Agrawal, T Imielinski, A N Swami, 1993. “Mining association rules between sets of items in large databases”, In “the Proceedings of the ACM SIGMOD Conference on management of data” pages 207-216, Washington D.C.
M A Bruno, E J Mufson, J Wuu, A C Cuello, “Increased matrix metalloproteinase 9 activity in mild cognitive impairment,” J Neuropathol Exp Neurol, epub November 2009.
G J Burbach, R Hellweg, C A Haas, D Del Turco, U Deicke, D Abramowski, M Jucker, M Staufenbiel, T Deller, “Induction of breain-derived neurotrophic factor in plaque-associated glial cells of aged APP23 transgenic mice,” J Neurosci 24:2421-30, 2004.
V W Delagarza, “Pharmacologic treatment of Alzheimer's disease: an update,” Clin Pharmacol 68:1365-72, 2003.
M Fahnestock, D Garzon, R M Holsinger, B Michalski, “Neurotrophic factors and Alzheimer's disease: are we focusing on the wrong molecule?” J Neural Transm Suppl 62:241-52, 2002.
Gottardo, “Statistical analysis of microarray data, A Bayesian approach,” Biostatistics 2001, 1,1, pp 1-37.
T Hastie, R Tibshirani, D Bolstein, P Brown, “Supervised harvesting of expression trees,” Genome Biol 2(1): reseach003, 2001.
N Helbecque, V Codron, D Cottel, P Amouyel, “An age effect on the association of common variants of ACE with Alzheimer's disease,” Neurosci Lett 461:181-4, 2009.
C W Huang, C C Lui, W N Chang, C H Lu, Y L Wang, C C Chang, “Elevated basal cortisol level predicts lower hippocampal volume and cognitive decline in Alzheimer's disease,” J Clin Neurosci, 16:1283-6, 2009.
D S Knopman, R C Petersen, S D Edland, R H Cha, W A Rocca, “The incidence of frontaltemporal lobar degeneration in Rochester, Minn., 1990 through 1994,” Neurology 62:506-8, 2004.
T Kohonen, “Self-organized formation of topologically correct feature maps,” Biol Cybernetics, 43:59-69, 1982.
A Mancinella, M Mancinella, G Carpinteri, A Bellomo, C Fossati, V Gianturco, A Iori, E Ettorre, G Troisi, V Marigliano, “Is there a relationship between high C-reactive protein (CRP) levels and dementia?” Arch Gerontol Geriatr, 49 Suppl 1:185-94, 2009.
A Mandavilli, “The amyloid code,” Nat Med 12:747-51, 2006.
E Masliah, M Mallory, M Alford, R Deteresa, T Saitoh, “PDGF is associated with neuronal and glial alterations of Alzheimer's disease,” Neurobiol Aging 16:549-56, 1995.
A C Paula-Lima, M A Tricerri, J Brito-Moreira, T R Bomfim, F F Oliveira, M H Magdesian, L T Grinberg, R Panizzutti, S T Ferreira, “Human apolipoprotein A-I binds amyloid-beta and prevents Abeta-induced neurotoxicity,” Int J Biochem Cell Biol, 41:1361-70, 2009.
D A Power, J Noel, R Collins, D O'Neill, “Circulating leptin levels and weight loss in Alzheimer's disease patients,” Dement Geriatr Cogn Disord 12:167-70, 2001.
S Ray, M Britschi, C Herbert, Y Takeda-Uchimura, A Boxer, K Blennow, L F Friedman, D R Galasko, M Jutel, A Karydas, J A Kaye, J Leszek, B L Miller, L Minthon, J F Quinn, G D Rabinovici, W H Robinson, M N Sabbagh, Y T So, D L Sparks, M Tabaton, J Tinklenberg, J A Yesavage, R Tibshirani, T Wyss-Coray, “Classification and prediction of clinical Alzheimer's diagnosis based on plasma signaling proteins,” Nat Med 13:1359-1362, 2007.
R O Roberts, Y E Geda, D S Knopman, B F Boeve, T J Christianson, V S Pankratz, I J Kullo, E G Tangalos, R J Ivnik, R C Petersen, “Association of C-reative protein with mild cognitive impairment,” Alzheimers Dement, 5:398-405, 2009.
F Song, A Poljak, G A Smythe, P Sachdev, “Plasma biomarkers for mild cognitive impairment and Alzheimer's disease,” Brain Res Rev, 61:69-80, 2009.
V G Tusher, R Tibshirani, G Chu, “Significance analysis of microarrays applied to ionizing radiation response,” Proc Natl Acad Sci, 98:5116-21, 2001.
H Wada, “Analyses of serum concentrations of apolipoproteins in the demented elderly,” Intern Med 39:220-2, 2000.
Worldwide web at expasy.org.
Worldwide web at stat.stanford.edu/˜tibs/PAM/index.html
J W Wright and J W Harding, “The brain RAS and Alzheimer's disease,” Exp Neurol epub September 2009.
K Zou, T Maeda, A Watanabe, J Liu, S Liu, R Oba, Y Satoh, H Komano, M Michikawa, “Abeta42-to-Abeta40- and angiotensin-converting activities in different domains of angiotensin-converting enzyme,” J Biol Chem 284:31914-20, 2009.
J Zhang, I Sokal, E R Peskind, J F Quinn, J Jankovic, C Kenney, K A Chung, SP Millard, J G Nutt, T J Montine, “CSF multianalyte profile distinguishes Alzheimer and Parkinson diseases,” Am J Clin Pathol 129:526-9, 2008.

Although the foregoing invention has been described in some detail by way of illustration and example for purposes of clarity of understanding, it will be apparent to those skilled in the art that certain changes and modifications may be practiced. Therefore, the descriptions and examples should not be construed as limiting the scope of the invention.

We claim:

1. A method of aiding diagnosis of Alzheimer's disease (“AD”), comprising:
a) measuring a level of each of at least three AD diagnosis biomarkers in a biological fluid sample in an individual, wherein the at least three AD diagnosis biomarkers are selected from the group consisting of: ACE (angiotensin converting enzyme or CD143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand)
b) implementing a scoring algorithm;
c) generating a score based on the measured levels of the AD diagnosis biomarkers; and
d) comparing the score with a cutoff score, wherein the diagnosis of AD is aided by determining whether the score is above, equal to, or below the cutoff score;
wherein the method has an accuracy of at least about 65% in classifying the individual as either having Alzheimer's disease or nondemented.

2. A computer implemented method of diagnosing or aiding diagnosis of Alzheimer's disease (“AD”) comprising: a) implementing a scoring algorithm; b) generating a score based on measured levels of a set of AD diagnosis biomarkers in a biological fluid sample in an individual; and c) comparing the score with a cutoff score, wherein the diagnosis of AD is made or aided by determining whether the score is above, equal to, or below the cutoff score, wherein the set of AD diagnosis biomarkers comprises at least three AD diagnosis biomarkers, wherein the at least three AD diagnosis biomarkers are selected from the group consisting of: ACE (angiotensin converting enzyme or CD 143), alpha-1-antitrypsin, Apo AI (apolipoprotein AI), Apo CIII (apolipoprotein CIII), Apo E (apolipoprotein E), CRP (C-reactive protein), cortisol, FGF-4 (fibroblast growth factor-4), MCP-3 (monocyte chemotactic protein-3), MMP-9 (matrix metalloproteinase-9), and TRAIL R3 (Receptor-3 for Tumor necrosis factor-related apoptosis-inducing ligand), and wherein the method has an accuracy of at least about 65% in classifying the individual as either having Alzheimer's disease or nondemented.

3. The method of claim 1, wherein the set of AD diagnosis biomarkers comprises at least four AD diagnosis biomarkers, wherein the at least four AD diagnosis biomarkers are selected from the group consisting of: ACE, alpha-1-antitrypsin, Apo AI, Apo CIII, Apo E, CRP, cortisol, FGF-4, MCP-3, MMP-9, and TRAIL R3.

4. The method of claim 1, wherein the set of AD diagnosis biomarkers comprises at least five AD diagnosis biomarkers, wherein the at least four AD diagnosis biomarkers are selected from the group consisting of: ACE, alpha-1-antitrypsin, Apo AI, Apo CIII, Apo E, CRP, cortisol, FGF-4, MCP-3, MMP-9, and TRAIL R3.

5. The method of claim 1, wherein the set of AD diagnosis biomarkers comprises at least six AD diagnosis biomarkers, wherein the at least four AD diagnosis biomarkers are selected from the group consisting of: ACE, alpha-1-antitrypsin, Apo AI, Apo CIII, Apo E, CRP, cortisol, FGF-4, MCP-3, MMP-9, and TRAIL R3.

6. The method of claim 1, wherein the set of AD diagosis biomakers comprises at least one of the following AD diagnosis biomarkers: ACE, cortisol, Apo E, MCP-3, and MMP-9.

7. The method of claim 1, wherein the set of AD diagosis biomakers comprises at least two of the following AD diagnosis biomarkers: ACE, cortisol, Apo E, MCP-3, and MMP-9.

8. The method of claim 1, wherein the set of AD diagosis biomakers comprises at least three of the following AD diagnosis biomarkers: ACE, cortisol, Apo E, MCP-3, and MMP-9.

9. The method of claim 1, wherein the set of AD diagnosis biomarkers comprises a set shown in Table 3.

10. The method of claim 1, wherein the method has an accuracy of at least about 67% in classifying the individual as either having Alzheimer's disease or nondemented.

11. The method of claim 1, wherein the method has an accuracy of at least about 69% in classifying the individual as either having Alzheimer's disease or nondemented.

12. The method of claim 1, wherein the method has an accuracy of at least about 71% in classifying the individual as either having Alzheimer's disease or nondemented.

13. The method of claim 1, wherein the cutoff score is generated by a scoring algorithm.

14. The method of claim 1, wherein the cutoff score is based on measured levels of the AD diagnosis biomarkers in a control population and a population having AD.

15. The method of claim 14, wherein the control population is a non-demented control (NDC) population.

16. The method of claim 14, wherein the control population is a healthy age-matched non-demented control (NDC) population.

17. The method of claim 1, wherein the biological fluid sample is a peripheral biological fluid sample.

18. The method of claim 17, wherein the peripheral biological fluid sample is blood, serum, or plasma.

19. The method of claim 1, wherein the individual is a human.

20. The method of claim 1, wherein the scoring algorithm comprises a method selected from the group consisting of: Significance Analysis of Microarrays, Tree Harvesting, CART, MARS, Self Organizing Maps, Frequent Item Set, Bayesian networks, Prediction Analysis of Microarray (PAM), SMO, Simple Logistic Regression, Logistic Regression, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, Ordinal Classifier, Sparse Linear Programming (SPLP), Sparse Logistic Regression (SPLR), Elastic NET, Support Vector Machine, Prediction of Residual Error Sum of Squares (PRESS), and combinations thereof.

21. The method of claim 20, wherein the method comprises Logistic Regression.

22. The method of claim 1, wherein the scoring algorithm is implemented by a computer.

23. The method of claim 1, wherein the score is determined by the formula: (e^{sum})/(1+e^{sum}), wherein the sum is determined by the formula: Intercept+ΣCoefficient(AD diagnosis biomarkerᵢ)*ln [AD diagnosis biomarkerᵢ+0.005], wherein i is 1 to N, N is the number of AD diagnosis biomarkers used, and [AD diagnosis biomarker] is the actual measured level of an AD diagnosis biomarker.

24. The method of claim 1, wherein the measuring is performed by a sandwich antibody array assay.

25. The method of claim 1, wherein the score is combined with one or more cognitive assessment tools and/or clinical observations in making the diagnosis or in aiding the diagnosis of AD.

26. The method of claim 1, wherein the measured levels are obtained by measuring the levels of the AD diagnosis biomarkers in the sample.

27. The method of claim 1, wherein the measured levels are obtained from a third party.
